(19) United States
(12) Patent Application Publication
(10) Pub. No.: US 2026/0076346 A1
(43) Pub. Date: Mar. 19, 2026

(54) IMMUNODEFICIENT NON-HUMAN ANIMALS FOR ASSESSING DRUG METABOLISM AND TOXICITY

(71) Applicant: UNIVERSITY OF DUNDEE, Dundee (GB)

(72) Inventors: Roland WOLF, Dundee (GB); Colin HENDERSON, Dundee (GB)

(21) Appl. No.: 19/109,590

(22) PCT Filed: Sep. 8, 2023
(86) PCT No.: PCT/GB2023/052320

§ 371 Date: Mar. 7, 2025

(30) Foreign Application Priority Data
Sep. 8, 2022 (GB) 2213141.1

(51) Int. Cl. A01K 67/0275; G01N 33/50

(52) U.S. Cl. CPC A01K 67/0275; G01N 33/5011; G01N 33/5088; A01K 2207/12; A01K 2207/15; A01K 2217/15; A01K 2227/105; A01K 2267/0331; A01K 2267/0337

(57) ABSTRACT

The present invention relates to an immunodeficient non-human animal where endogenous drug metabolising enzymes and transcription factors are substantially inactivated. In some aspects of the invention, the animal expresses human drug metabolising enzymes and associated transcription factors. There is also provided related methods of generating the animal of the invention. Additionally, there is provided methods of performing preclinical drug studies using the animal of the invention.

### FIELD OF THE INVENTION

The present invention relates to an immunodeficient non-human animal model of drug metabolism in which the endogenous drug metabolism system has been inactivated and in some aspects enzymes and transcription factors of the human drug metabolism system are expressed. The invention further relates to methods of generating the animal model and methods and uses of the animal model in xenograft drug studies and for other studies where immune deprived animals are required.

### BACKGROUND OF THE INVENTION

A significant hurdle in drug development is the transition from pre-clinical studies to human clinical trials. Currently, there is an overall failure rate in drug development of over 96%, with a staggering 90% of drugs failing during clinical development¹. At present, most pre-clinical metabolic and toxicity testing of drug candidates rely on laboratory animals and cell lines in vitro. None of these methods are completely reliable in predicting metabolism or toxicity in a human subject. Metabolic and toxicological data from animals can differ significantly from that obtained from a human subject due to species differences in the biochemical mechanisms involved. In addition, interpretation of data derived from in vitro human cell cultures or isolated human tissue studies can be problematic as they often fail to retain the same metabolic characteristics as they possess in vivo and do not have the physiological contact of a living animal.

Recently, a plethora of new targeted anti-cancer drugs (TAD) have been developed markedly improving progression free survival. Over 70 drugs targeted at protein kinases alone have obtained regulatory approval. Whilst there can be remarkable responses to targeted cancer treatment, invariably the cancer progresses as a consequence of diminished drug sensitivity and drug resistance.

To improve the outcomes of cancer treatment the application of more complex drug combinations is being explored by all drug companies. This generates a major pharmacological challenge in defining the optimal drug combination, the dosing regimen both in deciding the pathway components to be targeted and also which of the drugs inhibiting the same target may be most effective and free of side-effects. In addition, individuality between patients in drug exposure, determined by enzymes such as the cytochrome P450s, will also be a key factor in defining patient response when such therapies are used. The latter could involve drug/drug interactions of the targeted anti-tumour agents with each other and also with other drugs given concomitantly.

There are numerous examples where studies in vitro do not extrapolate to in vivo and where animal studies do not extrapolate to man. For example, a recent clinical study designed to establish whether changing the dosing regimen of dabrafenib and trametinib improved patient survival was based on xenograft experiments with vemurafenib (an alternative BRAFV600E inhibitor to dabrafenib) given individually which showed increased efficacy by introducing drug holidays. The clinical trial failed to show any improvement over the standard of care. This work demonstrates not only the issues with interspecies extrapolation but also the need for pharmacological principles in the design of clinical trials. There is an urgent need for novel model systems which improve extrapolation to man and improve therapeutic outcomes.

One major species difference between rodents and man is in the metabolism of drugs by the cytochrome P450 system. This generates differences in drug pharmacokinetics, in the metabolites produced, which can be pharmacologically active or toxic, and in the adaptive regulation of the P450 genes by the transcription factors CAR and PXR. It is therefore not surprising that the use of mouse models for drug efficacy studies often do not extrapolate to man. Suitably, there is a need for an animal model to investigate drug candidates in vivo, in the context of the human cytochrome P450 system and in the presence of human derived cells of interest in order to reduce the attrition rate of clinical trials and ultimately improve treatments for patients.

Transgenic animal models with deficiencies in murine drug metabolism are known in the art. However none of these models address the problem of the differences between the human and mouse P450 cytochrome systems and allow for studying patient-derived xenografts in a disease-specific context.

WO2009/109769 describes a transgenic animal model that has been humanised for transcription factors pregnane X receptor (PXR), the constitutive androstane receptor (CAR) and peroxisome proliferator activated receptor alpha (PPARa) and where the endogenous host animal genes have concomitantly inactivated.

WO2009/050484 describes a transgenic animal model where all members of the murine P450 cytochrome system have been inactivated but not replaced with human genes of equivalent function to those of the deleted murine P450 cluster. WO2005/074677 describes a cytochrome P450 reductase null mouse, where the mouse completely lacks P450-mediated metabolism in the liver. This model can be immune deprived to allow for assessing the contribution of human hepatocytes to drug metabolism in the model where P450 is inactive in the liver. However, it is a misconception that the liver is the only truly important tissue for drug metabolism. In reality, drug metabolism enzymes are also expressed in the small intestine, the lungs, placenta, and kidneys². The abrogation of function across all tissues is thus beneficial in order to study the effect of drug metabolism and accurately translate medicines to man.

The models known in the art additionally have problems with redundancy, wherein the cytochrome P450s in the remaining murine gene families can catalyse reactions being studied and compensatory changes resulting in the induced expression of murine P450s have been observed³. Furthermore, mice carrying individual humanisations are expensive to maintain and any individual line would not allow for evaluation of the complexities of the possible interactions between multiple human P450s expressed from multiple gene families. Finally, none of the models known in the art have entire abrogation of endogenous P450s across the all tissues and are immunodeficient to allow for patient-derived xenograft drug studies. One issue with such a genetically complex model is the viability of the offspring. Therefore, it was remarkable that the mouse model of the present invention was indeed viable.

The present invention described herein aims to address some of the problems described above. The models of the present invention may have endogenous drug metabolism enzymes and transcription factors substantially inactivated and may express major components of the human drug metabolism system, as well as lacking a functional adaptive immune system to allow for patient-derived xenograft drug metabolism studies. The models of the present invention are the most complex transgenic models ever made that allow for the study of human drug metabolism in vivo, but also in the context of patient-derived cells.

Importantly, the model is not limited to the field of oncology. The model has wide ranging applications such as investigating therapies in inflammatory or infectious diseases that are not possible in a model with a functioning immune system. The present invention aims reduce the extent of attrition in drug discovery but also allow for patient-tailored therapies across many clinical fields.

### SUMMARY OF THE INVENTION

In a first aspect of the present invention, there is provided an immunodeficient non-human animal wherein at least one endogenous drug metabolism enzyme has been substantially inactivated.

Suitably, in some embodiments, the activity of the at least one endogenous drug metabolism enzyme is at least 50%, at least 60%, at least 70%, at least 80%, at least 85%, at least 90%, at least 95%, at least 96%, at least 97%, at least 98%, at least 99% or 100% less active than the wild-type enzyme. In a preferred embodiment, the at least one endogenous drug metabolism enzyme has no activity. The term ‘no activity’ as referred to herein may mean that the enzyme does not perform its normal function or does not substantially perform its normal function. The skilled person will understand that the terms “inactivated”, “inactive” and “no activity” may be used interchangeably herein.

In one embodiment, the at least one endogenous drug metabolism enzyme is substantially inactivated in any one or more of the following tissues of the immunodeficient non-human animal of the invention: the liver, the small intestine, the lungs, the placenta, and/or kidneys. In a preferred embodiment, the at least one endogenous drug metabolism enzyme is substantially inactivated in all tissues of the immunodeficient non-human animal.

### Endogenous Drug Metabolism Genes

It is clear to the skilled person that the at least one endogenous drug metabolism enzyme of the invention as disclosed herein can be inactivated by any suitable method known in the art. In a preferred embodiment, the at least one endogenous drug metabolism enzyme is substantially inactivated by substantially inactivating at least one gene encoding a drug metabolism enzyme.

In one embodiment, there is provided an immunodeficient non-human animal wherein at least one gene encoding an endogenous drug metabolism enzyme is substantially inactivated.

Suitably, in some embodiments, at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, at least twenty-four, at least twenty-five, at least twenty-six, at least twenty-seven, at least twenty-eight, at least twenty-nine, at least thirty, at least thirty-one, at least thirty-two, at least thirty-three, at least thirty-four, at least thirty-five, at least thirty-six, at least thirty-seven, at least thirty-eight, at least thirty-nine, at least forty, at least fifty, or at least sixty genes encoding an endogenous drug metabolism enzyme are substantially inactivated in the non-human animal of the invention. In a preferred embodiment, a plurality of genes encoding an endogenous drug metabolism enzyme are substantially inactivated.

Suitably, in some embodiments the at least one endogenous gene encodes an enzyme of the endogenous cytochrome P450 monooxygenase system.

Accordingly, in some embodiments, the at least one endogenous gene encoding an endogenous drug metabolism enzyme is any one or more genes comprised in the Cyp1a, Cyp2a, Cyp2b, Cyp2c, Cyp2d, Cyp2e, Cyp3a or Cyp4a gene subfamilies.
The Cyp1a subfamily comprises: Cyp1a1, Cyp1a2 and functionally equivalent orthologues and homologues.
The Cyp2a subfamily comprises: Cyp2a4, Cyp2a5, Cyp2a12, Cyp2a22, Cyp2b2, Cyp2b3, Cyp2b12, Cyp2b15, Cyp2b21, Cyp2b31 and functionally equivalent orthologues and homologues.
The Cyp2c subfamily comprises: Cyp2c55, Cyp2c65, Cyp2c66, Cyp2c29, Cyp2c38, Cyp2c39, Cyp2c67, Cyp2c68, Cyp2c40, Cyp2c69, Cyp2c37, Cyp2c44, Cyp2c54, Cyp2c50, Cyp2c70, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2c81 and functionally equivalent orthologues and homologues.
The Cyp2d subfamily comprises: Cyp2d1, Cyp2d2, Cyp2d3, Cyp2d4, Cyp2d5 Cyp2d22, Cyp2d11, Cyp2d10, Cyp2d9, Cyp2d12, Cyp2d34, Cyp2d13, Cyp2d40, Cyp2d26, and functionally equivalent orthologues and homologues.
The Cyp2e subfamily comprises: Cyp2e1 and functionally equivalent orthologues and homologues.
The Cyp3a subfamily comprises: Cyp3a1, Cyp3a2, Cyp3a9, Cyp3a18, Cyp3a23, Cyp3a62, Cyp3a73, Cyp3a13, Cyp3a11, Cyp3a16, Cyp3a25, Cyp3a41, Cyp3a44, Cyp3a57, Cyp3a58-ps, Cyp3a59 and functionally equivalent orthologues and homologues.
The Cyp4a family comprises: Cyp4a10, Cyp4a12, Cyp4a14, Cyp4a29, Cyp4a30, Cyp4a32, Cyp4a1, Cyp4a2, Cyp4a3, Cyp4a8 and functionally equivalent orthologues and homologues.

In some embodiments, the at least one endogenous gene encoding an endogenous drug metabolism enzyme is any one or more genes comprised in the Cyp1a, Cyp2c, Cyp2d, Cyp2e or Cyp3a gene subfamilies or any functionally equivalent orthologues and homologues.

Suitably, in one embodiment, the at least one endogenous gene encodes any one or more of Cyp1a1 and Cyp1a2, or functionally equivalent orthologues and homologues.

In a further embodiment, the at least one endogenous gene encodes any one or more, suitably all, of Cyp2c55, Cyp2c65, Cyp2c66, Cyp2c29, Cyp2c38, Cyp2c39, Cyp2c67, Cyp2c68, Cyp2c40, Cyp2c69, Cyp2c37, Cyp2c44, Cyp2c54, Cyp2c50, Cyp2c70, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2c81, or functionally equivalent orthologues and homologues.

In one embodiment, the at least one endogenous gene encodes any one or more, suitably all, of Cyp2d1, Cyp2d2, Cyp2d3, Cyp2d4, Cyp2d5 Cyp2d22, Cyp2d11, Cyp2d10, Cyp2d9, Cyp2d12, Cyp2d34, Cyp2d13, Cyp2d40, Cyp2d26, or functionally equivalent orthologues and homologues.

In one embodiment, the at least one endogenous gene encodes Cyp2e1 or functionally equivalent orthologues and homologues.

In another embodiment, the at least one endogenous gene encodes any one or more, suitably all, of Cyp3a1, Cyp3a2, Cyp3a9, Cyp3a18, Cyp3a23, Cyp3a62, Cyp3a73, Cyp3a13, Cyp3a11, Cyp3a16, Cyp3a25, Cyp3a41, Cyp3a44, Cyp3a57, Cyp3a58-ps, Cyp3a59, and functionally equivalent orthologues and homologues.

In a further embodiment, the at least one endogenous gene encodes any one or more, suitably all, of Cyp1a1, Cyp1a2, Cyp2c55, Cyp2c65, Cyp2c66, Cyp2c29, Cyp2c38, Cyp2c39, Cyp2c67, Cyp2c68, Cyp2c40, Cyp2c69, Cyp2c37, Cyp2c44, Cyp2c54, Cyp2c50, Cyp2c70, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2d1, Cyp2d2, Cyp2d3, Cyp2d4, Cyp2d5 Cyp2d22, Cyp2d11, Cyp2d10, Cyp2d9, Cyp2d12, Cyp2d34, Cyp2d13, Cyp2d40, Cyp2d26, Cyp2e1, Cyp3a1, Cyp3a2, Cyp3a9, Cyp3a18, Cyp3a23, Cyp3a62, Cyp3a73, Cyp3a13, Cyp3a11, Cyp3a16, Cyp3a25, Cyp3a41, Cyp3a44, Cyp3a57, Cyp3a58-ps, Cyp3a59 or functionally equivalent orthologues and homologues.

In one embodiment, wherein the non-human animal is a mouse, the at least one endogenous gene encodes any one or more, suitably all, of Cyp1a1, Cyp1a2, Cyp2a4, Cyp2a5, Cyp2a12, Cyp2a22, Cyp2b9, Cyp2b10, Cyp2b13, Cyp2b19, Cy2b23, Cyp2c29, Cyp2c37, Cyp2c38, Cyp2c39, Cyp2c40, Cyp2c44, Cyp2c50, Cyp2c54, Cyp2c55, Cyp2c65, Cyp2c66, Cyp2c67, Cyp2c68, Cyp2c69, Cyp2c70, Cyp2d9, Cyp2d10, Cyp2d11, Cyp2d12, Cyp2d13, Cyp2d22, Cyp2d26, Cyp2d34, Cyp2d40, Cyp2e1, Cyp3a11, Cyp3a13, Cyp3a16, Cyp3a25, Cyp3a41, Cyp3a44, Cyp3a57, Cyp3a59, Cyp4a10, Cyp4a12, Cyp4a14, Cyp4a29, Cyp4a30 and Cyp4a32.

In another embodiment, wherein the non-human animal is a rat, the at least one endogenous gene encodes any one or more, suitably all, of Cyp1a1, Cyp1a2, Cyp2a2, Cyp2a3, Cyp2b2, Cyp2b3, Cyp2b12, Cyp2b15, Cyp2b21, Cyp2b31, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2c81, Cyp2c68, Cyp2c69, Cyp2c70, Cyp2d1, Cyp2d2, Cyp2d3, Cyp2d4, Cyp2d5, Cyp2e1, Cyp3a1, Cyp3a2, Cyp3a9, Cyp3a18, Cyp3a23, Cyp3a62, Cyp3a73, Cyp4a1, Cyp4a2, Cyp4a3 and Cyp4a8.

In some embodiments, at least one gene encoding endogenous transcription factors Car or Pxr are substantially inactivated in the immunodeficient non-human animal as described herein. In some embodiments, both Car and Pxr are substantially inactivated.

Suitably, in one embodiment, any one or more, suitably all, of Cyp1a, Cyp2c, Cyp2e, Cyp2d, or Cyp3a gene subfamilies and Car or functionally equivalent orthologues and homologues thereof are substantially inactivated in the immunodeficient non-human animal of the present invention.

Suitably, in another embodiment, any one or more, suitably all, of Cyp1a, Cyp2c, Cyp2e, Cyp2d, or Cyp3a gene subfamilies and Pxr or functionally equivalent orthologues and homologues thereof are substantially inactivated in the immunodeficient non-human animal of the present invention.

In yet another embodiment, any one or more of Cyp1a, Cyp2c, Cyp2e, Cyp2d, or Cyp3a gene subfamilies and Car and Pxr or functionally equivalent orthologues and homologues thereof are substantially inactivated in the immunodeficient non-human animal of the present invention.

In a further embodiment, any two, any three, any four or more of Cyp1a, Cyp2c, Cyp2e, Cyp2d, or Cyp3a gene subfamilies and Car or functionally equivalent orthologues and homologues thereof are substantially inactivated in the immunodeficient non-human animal of the present invention.

In a further embodiment, any two, any three, any four or more of Cyp1a, Cyp2c, Cyp2e, Cyp2d, or Cyp3a gene subfamilies and Pxr or functionally equivalent orthologues and homologues thereof are substantially inactivated in the immunodeficient non-human animal of the present invention.

In a further embodiment, any two, any three, any four or more of Cyp1a, Cyp2c, Cyp2e, Cyp2d, or Cyp3a gene subfamilies and Car and Pxr or functionally equivalent orthologues and homologues thereof are substantially inactivated in the immunodeficient non-human animal of the present invention.

In one embodiment, Cyp1a, Cyp2c, Cyp2d, and Cyp3a gene subfamilies and Pxr and Car are substantially inactivated in the immunodeficient non-human animal of the present invention.

In one embodiment, Cyp1a, Cyp2c, Cyp2e, Cyp2d, and Cyp3a gene subfamilies and Pxr and Car are substantially inactivated in the immunodeficient non-human animal of the present invention.

In another embodiment, Cyp1a1, Cyp1a2, Cyp2c29, Cyp2c37, Cyp2c38, Cyp2c39, Cyp2c40, Cyp2c44, Cyp2c50, Cyp2c54, Cyp2c55, Cyp2c65, Cyp2c66, Cyp2c67, Cyp2c68, Cyp2c69, Cyp2c70, Cyp2d9, Cyp2d10, Cyp2d11, Cyp2d12, Cyp2d13, Cyp2d22, Cyp2d26, Cyp2d34, Cyp2d40, Cyp3a11, Cyp3a13, Cyp3a16, Cyp3a25, Cyp3a41, Cyp3a44, Cyp3a57, Cyp3a59 and Pxr and Car are substantially inactivated in the immunodeficient non-human animal of the present invention. Suitably, in such embodiments, the non-human animal may be a mouse

In another embodiment, Cyp1a1, Cyp1a2, Cyp2c29, Cyp2c37, Cyp2c38, Cyp2c39, Cyp2c40, Cyp2c44, Cyp2c50, Cyp2c54, Cyp2c55, Cyp2c65, Cyp2c66, Cyp2c67, Cyp2c68, Cyp2c69, Cyp2c70, Cyp2d9, Cyp2d10, Cyp2d11, Cyp2d12, Cyp2d13, Cyp2d22, Cyp2d26, Cyp2d34, Cyp2d40, Cyp2e1, Cyp3a11, Cyp3a13, Cyp3a16, Cyp3a25, Cyp3a41, Cyp3a44, Cyp3a57, Cyp3a59 and Pxr and Car are substantially inactivated in the immunodeficient non-human animal of the present invention. Suitably, in such embodiments, the non-human animal may be a mouse.

In another embodiment, Cyp1a1, Cyp1a2, Cyp2a2, Cyp2a3, Cyp2b2, Cyp2b3, Cyp2b12, Cyp2b15, Cyp2b21, Cyp2b31, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2c81, Cyp2c68, Cyp2c69, Cyp2c70, Cyp2d1, Cyp2d2, Cyp2d3, Cyp2d4, Cyp2d5, Cyp3a1, Cyp3a2, Cyp3a9, Cyp3a18, Cyp3a23, Cyp3a62, Cyp3a73, Cyp4a1, Cyp4a2, Cyp4a3, Cyp4a8 and Pxr and Car are substantially inactivated in the immunodeficient non-human animal of the present invention. Suitably, in such embodiments, the non-human animal may be a rat.

In another embodiment, Cyp1a1, Cyp1a2, Cyp2a2, Cyp2a3, Cyp2b2, Cyp2b3, Cyp2b12, Cyp2b15, Cyp2b21, Cyp2b31, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2c81, Cyp2c68, Cyp2c69, Cyp2c70, Cyp2d1, Cyp2d2, Cyp2d3, Cyp2d4, Cyp2d5, Cyp2e1, Cyp3a1, Cyp3a2, Cyp3a9, Cyp3a18, Cyp3a23, Cyp3a62, Cyp3a73, Cyp4a1, Cyp4a2, Cyp4a3, Cyp4a8 and Pxr and Car are substantially inactivated in the immunodeficient non-human animal of the present invention. Suitably, in such embodiments, the non-human animal may be a rat.

It is evident to the skilled person that the immunodeficient non-human animal described herein may be homozygous for some or all of the substantially inactivated genes described herein. In some embodiments, the immunodeficient animal is heterozygous for some or all of the substantially inactivated genes described herein. The skilled person would understand that heterozygosity of an endogenous subfamily gene cluster refers to one functional allele of each gene contained within the subfamily and one substantially inactivated allele of each gene contained within the subfamily, unless otherwise specified.

Suitably, in one embodiment the immunodeficient non-human animal is homozygous for any combination (or all) of endogenous drug metabolism genes and transcription factor genes that have been substantially inactivated as described in the embodiments above.

In another embodiment, the immunodeficient non-human animal is heterozygous for any combination of endogenous drug metabolism genes and transcription factor genes that have been substantially inactivated as described in the embodiments above.

In some embodiments, it is preferable that the immunodeficient animal model retains one functional allele of the endogenous Cyp2c subfamily. Suitably, in some embodiments, the immunodeficient animal model is heterozygous for the endogenous Cyp2c subfamily gene cluster inactivation. Suitably, in some embodiments, wherein the immunodeficient animal model is heterozygous for the endogenous Cyp2c subfamily gene cluster inactivation, the animal retains one functional version of each gene in the Cyp2c subfamily and one substantially inactivated version of each gene in the Cyp2c subfamily. In another embodiment, the immunodeficient non-human animal is homozygous for the Cyp2c subfamily gene cluster inactivation except for Cyp2c44, wherein one functional allele is retained for Cyp2c44, i.e. the animal is heterozygous for Cyp2c44 inactivation. Suitably, in some embodiments the immunodeficient non-human animal retains one functional version of Cyp2c44 and one substantially inactivated version of Cypc44. In such embodiments where the immunodeficient animal model is heterozygous for the endogenous Cyp2c subfamily gene cluster inactivation, preferably the animal is female. Similarly, in embodiments where the immunodeficient animal model is homozygous for the endogenous Cyp2c subfamily gene cluster inactivation, preferably the animal is male.

In a most preferred embodiment, when the animal is male, the immunodeficient non-human animal is homozygous for any combination of endogenous drug metabolism genes and transcription factor genes substantially inactivated as described herein. In another preferred embodiment, when the animal is female, the immunodeficient non-human animal is homozygous for any combination of endogenous drug metabolism genes and transcription factor genes substantially inactivated as described herein, except for the endogenous Cyp2c subfamily where one functional allele is retained.

In any of the embodiments described herein, the at least one endogenous drug metabolism enzyme is substantially inactivated in some or all of the tissues of the immunodeficient animal. In some embodiments, the at least one gene encoding an endogenous drug metabolism enzyme is substantially inactivated in some or all of the tissues. Suitably, in some embodiments, transcription factors Car and/or Pxr, or the endogenous genes encoding Car and/or Pxr may also be substantially inactivated in some or all of the tissues of the immunodeficient animal of the present invention.

Suitably, in some embodiments, the at least one endogenous drug metabolism enzyme, the at least one gene encoding an endogenous drug metabolism enzyme, the transcription factors Car and/or Pxr, or the endogenous genes encoding Car and/or Pxr may be substantially inactivated in the in the liver, the small intestine, the lungs, the placenta, and/or kidneys. In an alternative embodiment, the at least one endogenous drug metabolism enzyme, the at least one gene encoding an endogenous drug metabolism enzyme, the transcription factors Car and/or Pxr, or the endogenous genes encoding Car and/or Pxr may be substantially inactivated in all tissues of the animal.

### Human Drug Metabolism System

As discussed above, a major challenge in translating drug candidates from animal studies to human treatments are the differences in the drug metabolism enzymes, particularly those of the cytochrome P450 system. These differences are known to cause dissimilarities in drug pharmacokinetics and metabolite production between animals and man and ultimately a high failure rate when translating drugs to man.

Suitably, in some embodiments of the present invention, the immunodeficient non-human animal as described herein additionally expresses at least one human drug metabolism enzyme. Accordingly, in some embodiments the animal is an immunodeficient transgenic non-human animal, suitably transgenic for at least one human drug metabolism enzyme gene.

Suitably, in some embodiments, the immunodeficient non-human animal expresses at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, at least twenty-four, at least twenty-five, at least twenty-six, at least twenty-seven, at least twenty-eight, at least twenty-nine, at least thirty, at least thirty-one, at least thirty-two, at least thirty-three, at least thirty-four, at least thirty-five, at least thirty-six, at least thirty-seven, at least thirty-eight, at least thirty-nine, at least forty, at least fifty, at least sixty, at least seventy, at least eighty, at least ninety, or at least one hundred human drug metabolism enzymes.

In a preferred embodiment, the immunodeficient non-human animal expresses a plurality of human drug metabolism enzymes. Suitably, in some embodiments, the human drug metabolism enzymes are one or more members of the human cytochrome P450 monooxygenase system.

Suitably, in some embodiments, the immunodeficient non-human animal expresses any one or more, suitably all, of the following human drug metabolism enzymes: CYP1A1, CYP1A2, CYP1B1, CYP2A6, CYP2B6, CYP2C8, CYP2C9, CYP2C18, CYP2C19, CYP2D6, CYP2E1, CYP3A4, CYP3A5, CYP3A7 and functional or allelic variants thereof.

In one embodiment, the immunodeficiency non-human animal expresses any one or more, suitably all, of the following human cytochrome P450 enzymes: CYP1A1, CYP1A2, CYP1B1, CYP2A6, CYP2A13, CYP2A7, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, CYP3A43, CYP4A11, CYP4A22, CYP4B1, CYP4F2, CYP4F3, CYP4F8, CYP4F11, CYP4F12, CYP4F22, CYP4V2, CYP4X1, or CYP4Z1, or any functional equivalents thereof.

Suitably, in some embodiments the immunodeficient non-human animal may express an entire human gene cluster. In one embodiment, the immunodeficient non-human animal expresses the human CYP1A cluster, the human CYP2C cluster, the human CYP3A cluster and/or the human CYP2D cluster.

In alternative embodiments, the immunodeficient non-human animal expresses any number of individual human genes, in any combination, from gene clusters CYP1A, CYP2C, CYP3A and/or CYP2D. Suitably, in one embodiment, the immunodeficient non-human animal expresses human CYP1A1 and/or CYP1A2. In another embodiment, the immunodeficient non-human animal expresses human CYP2C8, CYP2C9, CYP2C18 and/or CYP2C19. In another embodiment, the immunodeficient non-human animal expresses human CYP3A4, CYP3A5, CYP3A7 and/or CYP3A43. In another embodiment, the immunodeficient non-human animal expresses human CYP2D6.

In one embodiment, the immunodeficient non-human animal may express any one or more of the following human genes: CYP1A1, CYP1A2, CYP2C9, CYP2D6, CYP3A4 or CYP3A7 in any combination thereof. Suitably, in some embodiments the immunodeficient non-human animal expresses CYP1A1 and any one or more, suitably all, of CYP1A2, CYP2C9, CYP2D6, CYP3A4 and CYP3A7. In a further embodiment the immunodeficient non-human animal expresses CYP1A2 and any one or more, suitably all, of CYP1A1, CYP2C9, CYP2D6, CYP3A4 and CYP3A7. In yet another embodiment, the immunodeficient non-human animal expresses CYP2C9 and any one or more, suitably all, of CYP1A1, CYP1A2, CYP2D6, CYP3A4 and CYP3A7. In another embodiment, the immunodeficient non-human animal expresses CYP2D6 and any one or more, suitably all, of CYP1A1, CYP1A2, CYP2C9, CYP3A4 and CYP3A7. In another embodiment, the immunodeficient non-human animal expresses CYP3A4 and any one or more, suitably all, of CYP1A1, CYP1A2, CYP2C9, CYP2D6 and CYP3A7. In yet another embodiment, the immunodeficient non-human animal expresses CYP3A7 and any one or more, suitably all, of CYP1A1, CYP1A2, CYP2C9, CYP2D6 and CYP3A4.

In a preferred embodiment, the immunodeficient non-human animal expresses human CYP1A1, CYP1A2, CYP2C9, CYP2D6, CYP3A4 and CYP3A7.

Transcription factors CAR and PXR play are important regulators of xenobiotic responses. However, the adaptive regulation of the P450 genes by CAR and PXR are known to be different between humans and animal species such as rodents. Accordingly, it is advantageous in some embodiments of the present invention that the immunodeficient non-human animal of the present invention also express human CAR and/or PXR.

Suitably, in some embodiments, the immunodeficient non-human animal of the present invention expresses any of the human drug metabolism enzymes as described above and human CAR or PXR. In a preferred embodiment, the immunodeficient non-human animal of the present invention expresses any of the human drug metabolism enzymes as described above and both of human CAR and PXR.

In a most preferred embodiment, the immunodeficient non-human animal expresses human CYP1A1, CYP1A2, CYP2C9, CYP2D6, CYP3A4, CYP3A7, CAR, and PXR, or any functional variants thereof.

In some embodiments, the immunodeficient non-human animal has been humanised. By humanised in the context of the present invention, it is meant that the animal expresses a human drug metabolism enzyme. A humanised mouse may also express human transcription factors CAR and/or PXR. It is intended in some embodiments that the human drug metabolism enzymes and transcription factors replace the function of the endogenous drug metabolism enzyme and transcription factors. It will be understood by the skilled person that this does not require a one-to-one replacement. It is intended that any human gene or gene cluster that is functionally capable of replacing substantially the same, substantially similar or substantially identical function of the endogenous gene or enzyme or transcription factor, which is substantially inactivated, is suitable for humanising the animal.

Remarkably, the inventors have found that in some embodiments of the present invention, an immunodeficient non-human animal can comprise substantial inactivation of endogenous Cyp1a, Cyp2c, Cyp2d, and Cyp3a endogenous gene clusters and endogenous Car and Pxr transcription factors, totalling 35 endogenous drug metabolising enzymes being substantially inactivated. In some embodiments, the animal of the invention can also express human CYP1A1, CYP1A2, CYP2C9, CYP2D6, CYP3A4, CYP3A7, CAR and PXR. Even more remarkably, in some embodiments, the animal is also immunodeficient via the substantial inactivation of endogenous Rag2. To the inventors' knowledge, this is the most complex, viable, transgenic model ever made.

Suitably, in one embodiment of the present invention, endogenous Cyp1a, Cyp2c, Cyp2d, and Cyp3a subfamilies and Pxr and Car are substantially inactivated, and human CYP1A1, CYP1A2, CYP2C9, CYP2D6, CYP3A4, CYP3A7, CAR and PXR are expressed in the immunodeficient non-human animal of the present invention. Suitably, in another embodiment, endogenous Cyp1a, Cyp2c, Cyp2d, and Cyp3a subfamilies and Pxr and Car are substantially inactivated, endogenous Rag2 is substantially inactivated and human CYP1A1, CYP1A2, CYP2C9, CYP2D6, CYP3A4, CYP3A7, CAR and PXR are expressed in the immunodeficient non-human animal of the present invention. In such embodiments, the model may be referred to as an immunodeficient 8HUM model.

Suitably, in one embodiment of the present invention, endogenous Cyp1a, Cyp2e, Cyp2c, Cyp2d, and Cyp3a subfamilies and Pxr and Car are substantially inactivated, and human CYP1A1, CYP1A2, CYP2C9, CYP2D6, CYP3A4, CYP3A7, CAR and PXR are expressed in the immunodeficient non-human animal of the present invention. Suitably, in another embodiment, endogenous Cyp1a, Cyp2e, Cyp2c, Cyp2d, and Cyp3a subfamilies and Pxr and Car are substantially inactivated, endogenous Rag2 is substantially inactivated and human CYP1A1, CYP1A2, CYP2C9, CYP2D6, CYP3A4, CYP3A7, CAR and PXR are expressed in the immunodeficient non-human animal of the present invention. In such embodiments, the model may be referred to as an immunodeficient 8HUM model.

Suitably, in one embodiment of the present invention, endogenous Cyp1a1, Cyp1a2, Cyp2c29, Cyp2c37, Cyp2c38, Cyp2c39, Cyp2c40, Cyp2c44, Cyp2c50, Cyp2c54, Cyp2c55, Cyp2c65, Cyp2c66, Cyp2c67, Cyp2c68, Cyp2c69, Cyp2c70, Cyp2d9, Cyp2d10, Cyp2d11, Cyp2d12, Cyp2d13, Cyp2d22, Cyp2d26, Cyp2d34, Cyp2d40, Cyp2e1, Cyp3a11, Cyp3a13, Cyp3a16, Cyp3a25, Cyp3a41, Cyp3a44, Cyp3a57, Cyp3a59 and Pxr and Car are substantially inactivated, and human CYP1A1, CYP1A2, CYP2C9, CYP2D6, CYP3A4, CYP3A7, CAR and PXR are expressed in the immunodeficient non-human animal of the present invention. Suitably, in another embodiment, endogenous Cyp1a1, Cyp1a2, Cyp2c29, Cyp2c37, Cyp2c38, Cyp2c39, Cyp2c40, Cyp2c44, Cyp2c50, Cyp2c54, Cyp2c55, Cyp2c65, Cyp2c66, Cyp2c67, Cyp2c68, Cyp2c69, Cyp2c70, Cyp2d9, Cyp2d10, Cyp2d11, Cyp2d12, Cyp2d13, Cyp2d22, Cyp2d26, Cyp2d34, Cyp2d40, Cyp2e1, Cyp3a11, Cyp3a13, Cyp3a16, Cyp3a25, Cyp3a41, Cyp3a44, Cyp3a57, Cyp3a59 and Pxr and Car are substantially inactivated, endogenous Rag2 is substantially inactivated and human CYP1A1, CYP1A2, CYP2C9, CYP2D6, CYP3A4, CYP3A7, CAR and PXR are expressed in the immunodeficient non-human animal of the present invention. In such embodiments, the model may be referred to as an immunodeficient 8HUM model.

Suitably, in one embodiment of the present invention, endogenous Cyp1a1, Cyp1a2, Cyp2c29, Cyp2c37, Cyp2c38, Cyp2c39, Cyp2c40, Cyp2c44, Cyp2c50, Cyp2c54, Cyp2c55, Cyp2c65, Cyp2c66, Cyp2c67, Cyp2c68, Cyp2c69, Cyp2c70, Cyp2d9, Cyp2d10, Cyp2d11, Cyp2d12, Cyp2d13, Cyp2d22, Cyp2d26, Cyp2d34, Cyp2d40, Cyp3a11, Cyp3a13, Cyp3a16, Cyp3a25, Cyp3a41, Cyp3a44, Cyp3a57, Cyp3a59 and Pxr and Car are substantially inactivated, and human CYP1A1, CYP1A2, CYP2C9, CYP2D6, CYP3A4, CYP3A7, CAR and PXR are expressed in the immunodeficient non-human animal of the present invention. Suitably, in another embodiment, endogenous Cyp1a1, Cyp1a2, Cyp2c29, Cyp2c37, Cyp2c38, Cyp2c39, Cyp2c40, Cyp2c44, Cyp2c50, Cyp2c54, Cyp2c55, Cyp2c65, Cyp2c66, Cyp2c67, Cyp2c68, Cyp2c69, Cyp2c70, Cyp2d9, Cyp2d10, Cyp2d11, Cyp2d12, Cyp2d13, Cyp2d22, Cyp2d26, Cyp2d34, Cyp2d40, Cyp3a11, Cyp3a13, Cyp3a16, Cyp3a25, Cyp3a41, Cyp3a44, Cyp3a57, Cyp3a59 and Pxr and Car are substantially inactivated, endogenous Rag2 is substantially inactivated and human CYP1A1, CYP1A2, CYP2C9, CYP2D6, CYP3A4, CYP3A7, CAR and PXR are expressed in the immunodeficient non-human animal of the present invention. In such embodiments, the model may be referred to as an immunodeficient 8HUM model.

In an alternative embodiment, of the present invention, endogenous Cyp1a1, Cyp1a2, Cyp2a2, Cyp2a3, Cyp2b2, Cyp2b3, Cyp2b12, Cyp2b15, Cyp2b21, Cyp2b31, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2c81, Cyp2c68, Cyp2c69, Cyp2c70, Cyp2d1, Cyp2d2, Cyp2d3, Cyp2d4, Cyp2d5, Cyp3a1, Cyp3a2, Cyp3a9, Cyp3a18, Cyp3a23, Cyp3a62, Cyp3a73, Cyp4a1, Cyp4a2, Cyp4a3, Cyp4a8 and Pxr and Car are substantially inactivated, and human CYP1A1, CYP1A2, CYP2C9, CYP2D6, CYP3A4, CYP3A7, CAR and PXR are expressed in the immunodeficient non-human animal of the present invention. Suitably, in another embodiment, endogenous Cyp1a1, Cyp1a2, Cyp2a2, Cyp2a3, Cyp2b2, Cyp2b3, Cyp2b12, Cyp2b15, Cyp2b21, Cyp2b31, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2c81, Cyp2c68, Cyp2c69, Cyp2c70, Cyp2d1, Cyp2d2, Cyp2d3, Cyp2d4, Cyp2d5, Cyp3a1, Cyp3a2, Cyp3a9, Cyp3a18, Cyp3a23, Cyp3a62, Cyp3a73, Cyp4a1, Cyp4a2, Cyp4a3, Cyp4a8 and Pxr and Car are substantially inactivated, endogenous Rag2 is substantially inactivated and human CYP1A1, CYP1A2, CYP2C9, CYP2D6, CYP3A4, CYP3A7, CAR and PXR are expressed in the immunodeficient non-human animal of the present invention. In such embodiments, the model may be referred to as an immunodeficient 8HUM model.

In an alternative embodiment, of the present invention, endogenous Cyp1a1, Cyp1a2, Cyp2a2, Cyp2a3, Cyp2b2, Cyp2b3, Cyp2b12, Cyp2b15, Cyp2b21, Cyp2b31, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2c81, Cyp2c68, Cyp2c69, Cyp2c70, Cyp2d1, Cyp2d2, Cyp2d3, Cyp2d4, Cyp2d5, Cyp2e1, Cyp3a1, Cyp3a2, Cyp3a9, Cyp3a18, Cyp3a23, Cyp3a62, Cyp3a73, Cyp4a1, Cyp4a2, Cyp4a3, Cyp4a8 and Pxr and Car are substantially inactivated, and human CYP1A1, CYP1A2, CYP2C9, CYP2D6, CYP3A4, CYP3A7, CAR and PXR are expressed in the immunodeficient non-human animal of the present invention. Suitably, in another embodiment, endogenous Cyp1a1, Cyp1a2, Cyp2a2, Cyp2a3, Cyp2b2, Cyp2b3, Cyp2b12, Cyp2b15, Cyp2b21, Cyp2b31, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2c81, Cyp2c68, Cyp2c69, Cyp2c70, Cyp2d1, Cyp2d2, Cyp2d3, Cyp2d4, Cyp2d5, Cyp2e1, Cyp3a1, Cyp3a2, Cyp3a9, Cyp3a18, Cyp3a23, Cyp3a62, Cyp3a73, Cyp4a1, Cyp4a2, Cyp4a3, Cyp4a8 and Pxr and Car are substantially inactivated, endogenous Rag2 is substantially inactivated and human CYP1A1, CYP1A2, CYP2C9, CYP2D6, CYP3A4, CYP3A7, CAR and PXR are expressed in the immunodeficient non-human animal of the present invention. In such embodiments, the model may be referred to as an immunodeficient 8HUM model.

In some embodiments, wherein the immunodeficient non-human animal is heterozygous for the Cyp2c subfamily gene cluster inactivation, the animal is heterozygous for human CYP2C expression. Suitably, in such embodiments, the non-human animal retains one functional allele of the Cyp2c subfamily gene cluster. In some embodiments, wherein the animal is homozygous for Cyp2c subfamily gene cluster inactivation, except where heterozygosity is retained for endogenous Cyp2c44 gene inactivation, the animal is heterozygous for human CYP2C expression. Suitably, in such embodiments, the animal may be female. The skilled person would understand that heterozygosity of an endogenous subfamily gene cluster refers to one functional allele of each gene contained within the subfamily and one substantially inactivated allele of each gene contained within the subfamily, unless otherwise specified. Likewise, heterozygous human genes for example CYP2C refers to one functional allele of the human gene being expressed in the animal and one non-functional allele, unless otherwise described.

It will be understood by the skilled person that an immunodeficient non-human animal of the present invention is a transgenic animal in embodiments where at least one human drug metabolism gene or gene encoding a transcription factor is expressed by the animal.

### Immunodeficient Non-Human Animal

In some embodiments, an immunodeficient non-human animal may refer to any non-human animal, preferably a mammal. Suitably, in some embodiments, the genome of the immunodeficient non-human animal is altered by the transfer of a gene or genes from another species or breed, preferably a human gene or genes. Suitably, in such embodiments the animal is an immunodeficient transgenic non-human animal. Animals of the present invention include rodents such as a mouse (Mus musculus), a rat (Rattus), a hamster (Cricetinae), a guinea pig (Cavia porcellus), a gerbil (Meriones unguiculatus) and a rabbit (Oryctolagus cuniculus). Other suitable animals include pigs (Sus), dogs (Canis lupus familiaris) and non-human primates such as a Rhesus macaque (Macaca mulatta) or marmosets (Callithrix jacchus).

Suitably, in some embodiments the immunodeficient non-human animal is a mammal. In one embodiment the immunodeficient non-human animal is any one selected from: a pig, a rat, a rabbit, a guinea pig, a gerbil, a dog, a mouse, or a non-human primate. In a preferred embodiment the immunodeficient non-human animal is a mouse (Mus musculus). In another embodiment the animal of the invention is a non-human primate.

In some embodiments, the immunodeficient non-human animal as described herein has a deficiency in the innate and/or adaptive arms of the immune system. In some embodiments the immunodeficient non-human animal has a deficiency in both the innate and adaptive immune system. In a preferred embodiment, the animal of the invention has a deficient adaptive immune system. It is apparent to the person skilled in the art that any animal suitable for successful xenograft transplantation is suitable for the present invention. Immunodeficient non-human animals of the present invention include those that are genetically modified to render the immune system non-functional and/or animals that have been subjected to sub-lethal irradiation to produce mice with a non-functional immune system.

In one embodiment, the immunodeficient non-human animal has inactive Rag2. Suitably, in some embodiments Rag2 has a loss of function mutation. A loss of function mutation may comprise a deletion, insertion or substitution. In some embodiments Rag2 comprises a frameshift mutation. In some embodiments, Rag2 comprises a point mutation. Suitably, in some embodiments the immunodeficient non-human animal is homozygous (Rag2^{null/null}). In one embodiment, the immunodeficient non-human animal of the present invention does not have functional T and B cells. It will be apparent to the skilled person that any gene editing technique known in the art can be used to generate a mutated Rag2 animal. Suitably, in some embodiments, CRISPR Cas9 gene editing techniques are used introduce a Rag2 mutation in a non-human animal. In another embodiment, CRISPR Cas12a gene editing techniques are used to introduce a Rag2 mutation in a non-human animal.

In a further embodiment, the immunodeficient non-human animal may comprise a Prkdc^{scid} mutation. Suitably, the mutation may be a loss of function mutation. A loss of function mutation may comprise a deletion, insertion or substitution. In some embodiments Prkdc^{scid} comprises a frameshift mutation. In some embodiments, Prkdc^{scid} comprises a point mutation. Suitably, in some embodiments the immunodeficient non-human animal is homozygous (Prkdc^{scid/scid}). Methods to generate a Prkdc^{scid} animal are widely known in the art. For example, the use of zinc-finger nucleases to generate rats that lack the Prkdc gene (SCID) are described in Mashimo et al., 2012. It will be apparent to the skilled person that any gene editing technique can be used to generate a Prkdc^{scid} animal. In some embodiments, CRISPR Cas9 or Cas12a gene editing techniques are used to generate a non-human animal that comprises a Prkdc^{scid} mutation.

In some embodiments, the Prkdc^{scid} immunodeficient non-human animal of the present invention lacks functional T, B cells and natural killer (NK) cells.

In some embodiments, the immunodeficient non-human animal is an embryo, a neonate or an adult.

### Cells

In a second aspect, there is provided a cell isolated from the immunodeficient non-human animal according to any aspect and embodiment described herein. In some embodiments, the cell is a eukaryotic cell, preferably a mammalian cell, more preferably a murine cell. In some embodiments the cell is a germ cell or a somatic cell.

Suitably the cell is any one or more of the following: a hepatocyte, a keratinocyte, a pneumocyte, a brain cell e.g. a neuron, glial cell, or astrocyte, a podocyte, a sperm cell, an ovum, an immune cell and a stem cell.

Cells according to this aspect of the invention may be derived from the immunodeficient non-human animal using standard techniques, as will be clear to the skilled reader. Suitable methods are described in many standard laboratory manuals, such as Davis et al., Basic Methods in Molecular Biology (1986); Sambrook Molecular Cloning; A Laboratory Manual, Third Edition (2000); Ausubel et al., 1991 [supra]; Spector, Goldman & Leinwald, 1998).

In one embodiment, the cell according to the second aspect is a stem cell isolated from the immunodeficient non-human animal. In some embodiments, a stem cell isolated from the immunodeficient non-human animal may be pluripotent, or partially differentiated. Suitably, in some embodiments, a stem cell may be an adult stem cell or an embryonic stem cell, In some embodiments, a stem cell may be from a post-embryonic developmental stage e.g. foetal, neonatal, juvenile, or adult. In one embodiment, a stem cell isolated in this manner may be used to generate specific types of cells such as hepatocytes and neuronal cells. Such a cell also forms an aspect of the present invention.

In a further aspect, there is provided a method of performing ex vivo or in vitro drug studies, wherein the method comprises:
(i) providing a cell according to the second aspect; optionally wherein the cell is maintained in cell culture;
(ii) administering to the cell at least one test compound;
(iii) analysing at least one cellular characteristic.

In some embodiments, the at least one cellular characteristic is any one or more of viability, cell shape, granularity, motility, gene expression, intracellular protein expression, cell surface protein expression, enzyme activity, membrane potential, extracellular acidification rate, intracellular pH and/or mitochondrial metabolism.

### Methods of Generating an Immunodeficient Non-human Animal

According to a fourth aspect, there is provided a method of generating an immunodeficient non-human animal, wherein the method comprises substantially inactivating at least one endogenous drug metabolism enzyme in the immunodeficient non-human animal. In a preferred embodiment, the at least one endogenous drug metabolism enzyme is substantially inactivated by substantially inactivating at least one gene encoding a drug metabolism enzyme.

In one embodiment, the method comprises substantially inactivating at least one gene encoding an endogenous drug metabolism enzyme. Suitably, in some embodiments, the at least one endogenous gene encoding an endogenous drug metabolism enzyme is any one or more genes comprised in the Cyp1a, Cyp2c, Cyp2d, Cyp2e or Cyp3a gene subfamilies or any functionally equivalent orthologues and homologues.

Suitably, in some embodiments, the method comprises substantially inactivating at least one or more, suitably all, of the list comprising: Cyp1a1, Cyp1a2, Cyp2c55, Cyp2c65, Cyp2c66, Cyp2c29, Cyp2c38, Cyp2c39, Cyp2c67, Cyp2c68, Cyp2c40, Cyp2c69, Cyp2c37, Cyp2c44, Cyp2c54, Cyp2c50, Cyp2c70, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2d1, Cyp2d2, Cyp2d3, Cyp2d4, Cyp2d5 Cyp2d22, Cyp2d11, Cyp2d10, Cyp2d9, Cyp2d12, Cyp2d34, Cyp2d13, Cyp2d40, Cyp2d26, Cyp2e1, Cyp3a1, Cyp3a2, Cyp3a9, Cyp3a18, Cyp3a23, Cyp3a62, Cyp3a73, Cyp3a13, Cyp3a11, Cyp3a16, Cyp3a25, Cyp3a41, Cyp3a44, Cyp3a57, Cyp3a58-ps, Cyp3a59 or functionally equivalent orthologues and homologues.

Suitably, in one embodiment, the method comprises substantially inactivating any one or more of Cyp1a1 and Cyp1a2, or functionally equivalent orthologues and homologues.

In a further embodiment, the method comprises substantially inactivating any one or more, suitably all, of Cyp2c55, Cyp2c65, Cyp2c66, Cyp2c29, Cyp2c38, Cyp2c39, Cyp2c67, Cyp2c68, Cyp2c40, Cyp2c69, Cyp2c37, Cyp2c44, Cyp2c54, Cyp2c50, Cyp2c70, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2c81, or functionally equivalent orthologues and homologues.

In one embodiment, the method comprises substantially inactivating any one or more, suitably all, of Cyp2d1, Cyp2d2, Cyp2d3, Cyp2d4, Cyp2d5 Cyp2d22, Cyp2d11, Cyp2d10, Cyp2d9, Cyp2d12, Cyp2d34, Cyp2d13, Cyp2d40, Cyp2d26, or functionally equivalent orthologues and homologues.

In one embodiment, the method comprises substantially inactivating Cyp2e1 or functionally equivalent orthologues and homologues.

In another embodiment, the method comprises substantially inactivating Cyp3a1, Cyp3a2, Cyp3a9, Cyp3a18, Cyp3a23, Cyp3a62, Cyp3a73, Cyp3a13, Cyp3a11, Cyp3a16, Cyp3a25, Cyp3a41, Cyp3a44, Cyp3a57, Cyp3a58-ps, Cyp3a59, and functionally equivalent orthologues and homologues.

In some embodiments, the method comprises substantially inactivating endogenous transcription factors Car and/or Pxr. In a preferred embodiment, the method comprises substantially inactivating both endogenous Car and Pxr. In an even more preferred embodiment, the method comprises substantially inactivating at least one endogenous drug metabolism enzyme as described in embodiments above and substantially inactivating endogenous transcription factors Car and/or Pxr in an immunodeficient non-human animal.

Suitably, in one embodiment, the method comprises substantially inactivating any one or more of Cyp1a, Cyp2c, Cyp2e, Cyp2d, or Cyp3a gene subfamilies and Car or functionally equivalent orthologues and homologues thereof in the immunodeficient non-human animal of the present invention.

Suitably, in another embodiment, the method comprises substantially inactivating any one or more of Cyp1a, Cyp2c, Cyp2e, Cyp2d, or Cyp3a gene subfamilies and Pxr or functionally equivalent orthologues and homologues thereof in the immunodeficient non-human animal of the present invention.

In yet another embodiment, the method comprises substantially inactivating any one or more of Cyp1a, Cyp2c, Cyp2e, Cyp2d, or Cyp3a gene subfamilies and Car and Pxr or functionally equivalent orthologues and homologues thereof in the immunodeficient non-human animal of the present invention.

In a further embodiment, the method comprises substantially inactivating any two, any three, any four or more of Cyp1a, Cyp2c, Cyp2e, Cyp2d, or Cyp3a gene subfamilies and Car or functionally equivalent orthologues and homologues thereof in immunodeficient non-human animal of the present invention.

In a further embodiment, the method comprises substantially inactivating any two, any three, any four or more of Cyp1a, Cyp2c, Cyp2e, Cyp2d, or Cyp3a gene subfamilies and Pxr or functionally equivalent orthologues and homologues thereof in the immunodeficient non-human animal of the present invention.

In a further embodiment, the method comprises substantially inactivating any two, any three, any four or more of Cyp1a, Cyp2c, Cyp2e, Cyp2d, or Cyp3a gene subfamilies and Car and Pxr or functionally equivalent orthologues and homologues thereof in the immunodeficient non-human animal of the present invention.

In one embodiment, the method comprises substantially inactivating Cyp1a, Cyp2c, Cyp2d, and Cyp3a gene subfamilies and Pxr and Car in the immunodeficient non-human animal of the present invention.

In one embodiment, the method comprises substantially inactivating Cyp1a, Cyp2c, Cyp2d, Cyp2e and Cyp3a gene subfamilies and Pxr and Car in the immunodeficient non-human animal of the present invention.

In one embodiment, the method comprises substantially inactivating Cyp1a1, Cyp1a2, Cyp2c29, Cyp2c37, Cyp2c38, Cyp2c39, Cyp2c40, Cyp2c44, Cyp2c50, Cyp2c54, Cyp2c55, Cyp2c65, Cyp2c66, Cyp2c67, Cyp2c68, Cyp2c69, Cyp2c70, Cyp2d9, Cyp2d10, Cyp2d11, Cyp2d12, Cyp2d13, Cyp2d22, Cyp2d26, Cyp2d34, Cyp2d40, Cyp3a11, Cyp3a13, Cyp3a16, Cyp3a25, Cyp3a41, Cyp3a44, Cyp3a57, Cyp3a59 and Pxr and Car in the immunodeficient non-human animal of the present invention. Suitably, in such embodiments, the non-human animal may be a mouse.

In one embodiment, the method comprises substantially inactivating Cyp1a1, Cyp1a2, Cyp2c29, Cyp2c37, Cyp2c38, Cyp2c39, Cyp2c40, Cyp2c44, Cyp2c50, Cyp2c54, Cyp2c55, Cyp2c65, Cyp2c66, Cyp2c67, Cyp2c68, Cyp2c69, Cyp2c70, Cyp2d9, Cyp2d10, Cyp2d11, Cyp2d12, Cyp2d13, Cyp2d22, Cyp2d26, Cyp2d34, Cyp2d40, Cyp2e1, Cyp3a11, Cyp3a13, Cyp3a16, Cyp3a25, Cyp3a41, Cyp3a44, Cyp3a57, Cyp3a59 and Pxr and Car in the immunodeficient non-human animal of the present invention. Suitably, in such embodiments, the non-human animal may be a mouse.

In another embodiment, the method comprises substantially inactivating Cyp1a1, Cyp1a2, Cyp2a2, Cyp2a3, Cyp2b2, Cyp2b3, Cyp2b12, Cyp2b15, Cyp2b21, Cyp2b31, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2c81, Cyp2c68, Cyp2c69, Cyp2c70, Cyp2d1, Cyp2d2, Cyp2d3, Cyp2d4, Cyp2d5, Cyp3a1, Cyp3a2, Cyp3a9, Cyp3a18, Cyp3a23, Cyp3a62, Cyp3a73, Cyp4a1, Cyp4a2, Cyp4a3, Cyp4a8 and Pxr and Car in the immunodeficient non-human animal of the present invention. Suitably, in such embodiments, the non-human animal may be a rat.

In another embodiment, the method comprises substantially inactivating Cyp1a1, Cyp1a2, Cyp2a2, Cyp2a3, Cyp2b2, Cyp2b3, Cyp2b12, Cyp2b15, Cyp2b21, Cyp2b31, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2c81, Cyp2c68, Cyp2c69, Cyp2c70, Cyp2d1, Cyp2d2, Cyp2d3, Cyp2d4, Cyp2d5, Cyp2e1, Cyp3a1, Cyp3a2, Cyp3a9, Cyp3a18, Cyp3a23, Cyp3a62, Cyp3a73, Cyp4a1, Cyp4a2, Cyp4a3, Cyp4a8 and Pxr and Car in the immunodeficient non-human animal of the present invention. Suitably, in such embodiments, the non-human animal may be a rat.

The at least one endogenous drug metabolism enzyme and transcription factors Car and Pxr may be substantially inactivated by any suitable means known in the art. In some embodiments, the method may comprise substantially inactivating the at least one endogenous drug metabolism enzyme and/or transcription factors Car and Pxr by complete deletion of the coding sequence of the gene from the immunodeficient non-human animal's genome. Alternatively, in some embodiments substantial inactivation may be accomplished by mutation of the coding sequence, either by way of insertion, deletion or substitution of other sequences. For example, in some embodiments one or more mutations (such as frameshift mutations) may be generated such that any resulting RNA transcript codes for a non-functional or truncated protein. In an alternative embodiment, an insertion may be made into the chromosomal sequence encoding the endogenous drug metabolism enzyme and endogenous transcription factors Car and Pxr. Similarly, in some embodiments a sequence may be exchanged with an endogenous gene sequence that is being substantially inactivated, such as a selection or marker sequence that can be used as the basis for screening for successful mutants. One such strategy has been devised by Wallace et al., albeit in the context of gene exchange, although this is applicable to the method of the present invention. This method envisages an exchange of sequence between host chromosome and a BAC or YAC vector, such that two intermolecular homologous recombination events are required for the vector-based replacement sequence to replace the endogenous genomic sequence.

In one embodiment of the method described herein, a mechanism of homologous recombination is used to exchange an endogenous gene encoding the at least one drug metabolism enzyme or transcription factors Car and Pxr for an alternative sequence not present in the endogenous sequence. Such a method preferably comprises the steps of: a) incorporating a pair of site-specific recombination sites into the immunodeficient animal's chromosome by homologous recombination such that the target endogenous gene that is to be replaced is flanked on each side by a recombination site; and b) effecting recombination between the site-specific recombination sites such that the endogenous target gene is excised from the chromosome, replaced by a residual site-specific recombination site.

Suitably, in some embodiments the method comprises a further step of introducing at least one DNA sequence encoding at least one human drug metabolising enzyme into the immunodeficient non-human animal. In such embodiments, the skilled person would understand that the at least one human drug metabolising enzyme is functional and the endogenous drug metabolism enzyme is substantially inactive.

Alternatively, in some embodiments, the method comprises a further step of introducing at least one DNA sequence encoding at least one human drug metabolising enzyme and at least one DNA sequence encoding human CAR or PXR, into the immunodeficient non-human animal. In a most preferred embodiment, at least one DNA sequence encoding at least one human drug metabolising enzyme and a DNA sequence encoding human CAR and a DNA sequence encoding human PXR are introduced into the immunodeficient non-human animal of the invention. In such embodiments, the skilled person would understand that the at least one human drug metabolising enzyme and the human CAR and/or PXR are functional and the endogenous drug metabolism enzyme and endogenous Car and/or Pxr are substantially inactive.

In some embodiments, the method comprises introducing a plurality of DNA sequences encoding a plurality of human drug metabolising enzymes in the immunodeficient non-human animal. Suitably, in such embodiments the method may further comprise introducing at least one DNA sequence encoding human CAR and/or PXR transcription factors. In a most preferred embodiment, the method comprises introducing a plurality of DNA sequences encoding a plurality of human drug metabolising enzymes and a DNA sequence encoding human CAR and a DNA sequence encoding human PXR into the immunodeficient non-human animal of the invention.

Suitably, in some embodiments of the method, the at least one DNA sequence encoding a human drug metabolism enzyme, encodes any member of the human cytochrome P450 monooxygenase system.

Suitably, in some embodiments of the method, the at least one DNA sequence encoding a human drug metabolism enzyme, encodes at least one or more, suitably all, of the following human drug metabolism enzymes: CYP1A1, CYP1A2, CYP1B1, CYP2A6, CYP2A13, CYP2A7, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, CYP3A43, CYP4A11, CYP4A22, CYP4B1, CYP4F2, CYP4F3, CYP4F8, CYP4F11, CYP4F12, CYP4F22, CYP4V2, CYP4X1, or CYP4Z1, or any functional equivalents thereof.

In one embodiment of the method, the at least one DNA sequence encoding a human drug metabolism enzyme, encodes the human CYP1A cluster, the human CYP2C cluster, the human CYP3A cluster and/or the human CYP2D cluster as described herein.

In alternative embodiments, the at least one DNA sequence encoding a human drug metabolism enzyme encodes any individual gene from gene clusters CYP1A, CYP2C, CYP3A and/or CYP2D as described above. Suitably, in one embodiment, the at least one DNA sequence encodes human CYP1A1 and/or CYP1A2. In another embodiment the at least one DNA sequence encodes human CYP2C8, CYP2C9, CYP2C18 and/or CYP2C19. In another embodiment, the at least one DNA sequence encodes human CYP3A4, CYP3A5, CYP3A7 and/or CYP3A43. In another embodiment, the at least one DNA sequence encodes human CYP2D6.

In one embodiment, the at least one DNA sequence encodes any one or more of the following human genes: CYP1A1, CYP1A2, CYP2C9, CYP2D6, CYP3A4 or CYP3A7 in any combination thereof. Suitably, in some embodiments the at least one DNA sequence encodes CYP1A1 and any one or more, suitably all, of CYP1A2, CYP2C9, CYP2D6, CYP3A4 and CYP3A7. In a further embodiment, the at least one DNA sequence encodes CYP1A2 and any one or more, suitably all, of CYP1A1, CYP2C9, CYP2D6, CYP3A4 and CYP3A7. In yet another embodiment, the at least one DNA sequence encodes CYP2C9 and any one or more, suitably all, of CYP1A1, CYP1A2, CYP2D6, CYP3A4 and CYP3A7. In another embodiment, the at least one DNA sequence encodes CYP2D6 and any one or more, suitably all, of CYP1A1, CYP1A2, CYP2C9, CYP3A4 and CYP3A7. In another embodiment, the at least one DNA sequence encodes CYP3A4 and any one or more, suitably all, of CYP1A1, CYP1A2, CYP2C9, CYP2D6 and CYP3A7. In yet another embodiment, the at least one DNA sequence encodes CYP3A7 and any one or more, suitably all, of CYP1A1, CYP1A2, CYP2C9, CYP2D6 and CYP3A4.

In one embodiment, the method further comprises introducing a plurality of DNA sequences encoding human CYP1A1, CYP1A2, CYP2C9, CYP2D6, CYP3A4 and CYP3A7. In another embodiment, the method further comprises introducing a plurality of DNA sequences encoding human CYP1A1, CYP1A2, CYP2C9, CYP2D6, CYP3A4, CYP3A7, PXR and CAR.

Suitably, in a most preferred embodiment, there is provided a method of generating an immunodeficient non-human animal, wherein the method comprises;
(i) substantially inactivating endogenous gene clusters Cyp1a, Cyp2c, Cyp2d, and Cyp3a and endogenous Car and Pxr in an immunodeficient non-human animal;
(ii) introducing one or more, suitably a plurality of, DNA sequences encoding human CYP1A1, CYP1A2, CYP2C9, CYP2D6, CYP3A4, CYP3A7, PXR and CAR in the immunodeficient non-human animal.

Suitably, in another preferred embodiment, there is provided a method of generating an immunodeficient non-human animal, wherein the method comprises;
(i) substantially inactivating endogenous gene clusters Cyp1a, Cyp2c, Cyp2e, Cyp2d, and Cyp3a and endogenous Car and Pxr in an immunodeficient non-human animal;
(ii) introducing one or more, suitably a plurality of, DNA sequences encoding human CYP1A1, CYP1A2, CYP2C9, CYP2D6, CYP3A4, CYP3A7, PXR and CAR in the immunodeficient non-human animal.

Suitably, in one embodiment, the method comprises substantially inactivating at least 33 or at least 34, endogenous cytochrome P450 monooxygenase system genes as described herein, optionally wherein Car and Pxr are also substantially inactivated. Suitably, the at least 33 or at least 34 endogenous cytochrome P450 monooxygenase system genes are selected from Cyp1a1, Cyp1a2, Cyp2c29, Cyp2c37, Cyp2c38, Cyp2c39, Cyp2c40, Cyp2c44, Cyp2c50, Cyp2c54, Cyp2c55, Cyp2c65, Cyp2c66, Cyp2c67, Cyp2c68, Cyp2c69, Cyp2c70, Cyp2d9, Cyp2d10, Cyp2d11, Cyp2d12, Cyp2d13, Cyp2d22, Cyp2d26, Cyp2d34, Cyp2d40, Cyp3a11, Cyp3a13, Cyp3a16, Cyp3a25, Cyp3a41, Cyp3a44, Cyp3a57, Cyp3a59, or selected from Cyp1a1, Cyp1a2, Cyp2a2, Cyp2a3, Cyp2b2, Cyp2b3, Cyp2b12, Cyp2b15, Cyp2b21, Cyp2b31, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2c81, Cyp2c68, Cyp2c69, Cyp2c70, Cyp2d1, Cyp2d2, Cyp2d3, Cyp2d4, Cyp2d5, Cyp3a1, Cyp3a2, Cyp3a9, Cyp3a18, Cyp3a23, Cyp3a62, Cyp3a73, Cyp4a1, Cyp4a2, Cyp4a3, and Cyp4a8.

Suitably, in one embodiment, the method comprises substantially inactivating at least 33, 34 or 35, endogenous cytochrome P450 monooxygenase system genes as described herein, optionally wherein Car and Pxr are also substantially inactivated. Suitably, the at least 33, 34 or 35 endogenous cytochrome P450 monooxygenase system genes are selected from Cyp1a1, Cyp1a2, Cyp2c29, Cyp2c37, Cyp2c38, Cyp2c39, Cyp2c40, Cyp2c44, Cyp2c50, Cyp2c54, Cyp2c55, Cyp2c65, Cyp2c66, Cyp2c67, Cyp2c68, Cyp2c69, Cyp2c70, Cyp2d9, Cyp2d10, Cyp2d11, Cyp2d12, Cyp2d13, Cyp2d22, Cyp2d26, Cyp2d34, Cyp2d40, Cyp2e1, Cyp3a11, Cyp3a13, Cyp3a16, Cyp3a25, Cyp3a41, Cyp3a44, Cyp3a57, Cyp3a59, or selected from Cyp1a1, Cyp1a2, Cyp2a2, Cyp2a3, Cyp2b2, Cyp2b3, Cyp2b12, Cyp2b15, Cyp2b21, Cyp2b31, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2c81, Cyp2c68, Cyp2c69, Cyp2c70, Cyp2d1, Cyp2d2, Cyp2d3, Cyp2d4, Cyp2d5, Cyp2e1, Cyp3a1, Cyp3a2, Cyp3a9, Cyp3a18, Cyp3a23, Cyp3a62, Cyp3a73, Cyp4a1, Cyp4a2, Cyp4a3, and Cyp4a8.

In one embodiment of the method, where the immunodeficient non-human animal is male, both alleles encoding the endogenous drug metabolising enzyme and/or transcription factors Car and Pxr are substantially inactivated, i.e. the animal is homozygous for genes that are substantially inactivated. In an alternative embodiment, where the non-human animal is female, both alleles encoding the endogenous drug metabolising enzyme and/or transcription factors Car and Pxr are substantially inactivated, except one allele for Cyp2c is retained.

Suitably, in some embodiments, the method further comprises introducing at least five human cytochrome P450 monooxygenase system genes and human CAR and PXR. In a preferred embodiment, the at least five human cytochrome P450 monooxygenase system genes are CYP1A1, CYP1A2, CYP2C9, CYP2D6, CYP3A4, and CYP3A7.

In some embodiments, it may be desirable to substantially inactivate endogenous Cyp2b genes and to introduce human CYP2B6. In another embodiment, it may be desirable to substantially inactivate endogenous Cyp2b and to introduce human CYP2C8, CYP2C19 and CYP2A6 genes. In some embodiments, it may be desirable to transiently express human CYP2B6 in the non-human immunodeficient animal. In another embodiment, it may be desirable to transiently express human CYP2C8, CYP2C19 and CYP2A6 genes in the non-human immunodeficient animal. Suitably, in some embodiments, human CYP2B6 is transiently expressed in any aspect or embodiment as described herein. Suitably, in some embodiments, human CYP2C8, CYP2C19 and CYP2A6 are transiently expressed in any aspect or embodiment as described herein. Where human CYP2B6, CYP2C8, CYP2C19 and/or CYP2A6 are expressed in the non-human immunodeficient animal in addition to the human drug metabolism genes as described herein, the animal may be described as “further humanised”.

In any one of the embodiments of the method described herein, endogenous Rag2 may be substantially inactive in the immunodeficient non-human animal.

Generally, the expressed human drug metabolism gene and/or transcription factor gene will share a degree of homology with the endogenous gene with which it is equivalent. Preferably, in some embodiments, the degree of homology will be greater than 30%, greater than 40%, greater than 50%, greater than 60%, greater than 70%, greater than 80%, greater than 90%, greater than 95%, greater than 96%, greater than 97%, greater than 98% or even greater than 99%.

In some embodiments, the present invention attempts to mirror the in vivo situation by providing a replacement gene in its entirety where this is possible. Suitably, in some embodiments, intron-exon junctions may be retained as in the natural system so that splicing events can happen exactly as in the natural situation. Where it is not simple to transpose the entire gene into a transgenic system, some embodiments of the invention use a combination of cDNA and genomic DNA in its constructs so that important intron-exon boundaries, where the majority of splicing events occur, are retained.

Accordingly, in some embodiments where it is known that the majority of splice variants occur as a result of splicing variation within a particular intron, this intron is preferably incorporated as genomic DNA in the construct, while less influential intronic sequences are not retained. This has the result that levels of functional mRNA and functional protein mirror the levels that are found in vivo in response to exposure to a particular drug or drug combination. Suitably, in some embodiments, the human drug metabolic enzyme or transcription factor is expressed at substantially the same, similar or identical levels to that of the endogenous drug metabolism enzyme or transcription factor.

Accordingly, in some embodiments, cDNA or genomic sequences are used to express any human drug metabolism enzymes or transcription factors as discussed herein. In another embodiment, a combination of cDNA and genomic sequences are used to express any human drug metabolism enzymes or transcription factors as discussed herein in the immunodeficient transgenic non-human animal of the present invention. For example, in the case of a transgenic animal expressing the human PXR gene, due to the large size of more than 35 kb of the human PXR gene, the intron-exon structure between exons 4 and 6 is preferably maintained (see WO2006/064197), since most splice variants are observed in this genomic region and is within the ligand-binding domain. This advantageously retains the sequence where most splice variants are observed and is conveniently located within the ligand-binding domain.

In some preferred embodiments, complete genomic DNA sequences are used to express any human drug metabolism enzymes or transcription factors as discussed herein in the immunodeficient non-human animal of the present invention. In the case of a transgenic animal expressing the human CAR gene, the relatively small size of the human CAR, which comprises roughly 7 kb from exon 2-9, makes it simple to retain the complete genomic structure in the targeting vector. Suitably, in one embodiment, the construct should preferably retain the intron-exon structure between exons 2 and 9 (see WO2006/064197). This advantageously retains the complete genomic structure within the targeting vector and permits coverage of all splice variants of human CAR. In a preferred embodiment, the genomic human CAR DNA sequence is fused to the translational start site of the endogenous Car gene of the immunodeficient transgenic non-human animal. In some embodiments, the human CAR sequence then contains all genomic sequences of exons 1-9. Suitably, in some embodiments the 5′ and 3′UTRs may be human or may be retained from the endogenous genome, optionally wherein all other parts of the coding sequences of the endogenous CAR gene can be deleted.

It will be understood by the skilled person that any genetic engineering technique known in the art can be used to introduce human genes as described herein into a non-human animal or to substantially inactivate the non-human animal genes such as Rag2, Prkdc^{scid} and/or any endogenous drug metabolism enzyme or transcription factor as described herein. In one embodiment, a Type II CRISPR system is a preferred method of modifying the non-human animal. Suitably, in some embodiments a Type II CRISPR Cas9 system is used to substantially inactivate at least one endogenous non-human animal drug metabolism gene. In another embodiment, a Type II CRISPR Cas9 system is used to introduce at least one human drug metabolism gene. In another embodiment, a Type II CRISPR Cas9 system is used to substantially inactivate at least one non-human animal drug metabolism gene and to introduce at least one human drug metabolism gene. In yet another embodiment, a Type II CRISPR Cas9 system is used to substantially inactivate endogenous Rag2. In one embodiment, a Type II CRISPR Cas9 system is used to substantially inactivate endogenous Prkdc^{scid}. Suitably, in some embodiments a Type II CRISPR Cas9 system is used to substantially inactivate a plurality of endogenous drug metabolism genes and transcription factor genes (e.g. Car and/or Pxr). In another embodiment, a Type II CRISPR Cas9 system is used to introduce a plurality of human genes, suitably human drug metabolism genes, CAR and/or PXR transcription factors. In one embodiment, a Type II CRISPR Cas9 system is used to substantially inactivate plurality of endogenous drug metabolism genes and transcription factors genes as described herein and to introduce human a plurality of human drug metabolism genes and transcription factor genes as described herein. It will be understood by the skilled person that Type II CRISPR Cas9 system may be replaced with any other suitable CRISPR system, for example, Class II type V CRISPR-Cas12a.

The skilled person will understand that human DNA may be introduced into the genome using transient expression for example using adenoviral or AAV approaches. Suitably, in some embodiments a human promoter or a heterologous promoter e.g. the albumin promoter may be used.

Nucleic acid sequences encoding any one or more human cytochrome drug metabolism enzyme gene, human CAR, and/or human PXR genes of the invention may be provided as expression constructs in the form of a plasmid, vector, transcription or expression cassette which comprises at least one nucleic acid as described above operably liked to one or more expression control sequences, e.g. a promoter, an enhancer, a poly-A sequence or an intron. The expression control sequences may be constitutive or regulatable.

Suitably, in some embodiments the expression of a human drug metabolism gene and/or human transcription factor as described herein may be driven by the human promoter or by the endogenous non-human animal promoter. In one embodiment, human CYP1A1 is driven by the endogenous non-human animal Cyp1a1 promoter. In another embodiment, human CYP1A2, is driven by the endogenous non-human animal Cyp1a2 promoter. In another embodiment, human CAR, is driven by the endogenous non-human animal Car promoter. In another embodiment, human PXR, is driven by the endogenous non-human animal Pxr promoter. Suitably, in one embodiment expression of human CYP2C9 is driven by human CYP2C9 promoter in the non-human immunodeficient animal. In another embodiment expression of human CYP2D6 is driven by human CYP2D6 promoter in the non-human immunodeficient animal. In another embodiment expression of human CYP3A4 is driven by human CYP3A4 promoter in the non-human immunodeficient animal. In another embodiment expression of human CYP3A7 is driven by human CYP3A7 promoter in the non-human immunodeficient animal. In another embodiment, expression of human CYP2C9 is driven by albumin promoter in the non-human immunodeficient animal. Suitably, in one embodiment expression of human CYP2C9, CYP2D6, CYP3A4, CYP3A7 is driven by the corresponding human promoter, human CYP2C9 is driven by albumin promoter and human CYP1A1, CYP1A2, CAR and PXR are driven by the respective endogenous promoter in the non-human immunodeficient animal of the present invention.

Suitably, the expression cassette may comprise a selection marker. Suitably, a selection marker is a positive selection gene such as a neomycin resistance gene. Suitably, a selection marker is a negative selection gene such as thymidine kinase, diphtheria toxin fragment A, or in such examples where the positive selection marker is flanked by loxP sites, Cre recombinase gene (Cre) is suitable. In one embodiment, the expression cassette comprises a fluorescent marker gene such as green fluorescent protein (GFP), yellow fluorescent protein (YFP), red fluorescent protein (RFP), mCherry, and/or luciferase. Suitably, the expression cassette may be provided in a vector.

### Drug Metabolism Studies

As discussed above, to improve the outcomes of diseases such as cancer, the application of more complex drug combinations is being explored. However, combination therapies have an increased risk of drug-drug interactions that can result in negative effects on drug efficacy and the increased likelihood of side effects. This generates a major pharmacological challenge in defining the optimal drug combination, the dosing regimen both in deciding the pathway components to be targeted and also which of the drugs inhibiting the same target may be most effective and free of side-effects. In addition, individuality between patients in drug exposure, determined by enzymes such as the cytochrome P450s, are also a key factor in defining patient response when such therapies are used.

The immunodeficient animal of the present invention aims to address some of these challenges. The immunodeficient non-human animal of the present invention may express human drug metabolising enzymes and transcription factors Car and Pxr but is also suitable for patient xenografts therefore allows for more accurate extrapolation of the efficacy of complex drug combinations, the potential for drug-drug interactions and predicted dosing schedules from animal to man. This aims to reduce the attrition rate of drug development and, importantly, allows optimal drug combinations to be investigated in a patient specific context.

Suitably, an immunodeficient non-human animal of the invention or a cell derived therefrom may be used as a model system for determining the metabolism of drugs or other xenobiotic compounds in other organisms, particularly the human.

Accordingly, in an further aspect, there is provided a method of performing preclinical drug studies, wherein the method comprises:
(i) providing an immunodeficient non-human animal according to the first aspect or fourth aspect;
(ii) administering at least one test compound to the animal;
(iii) obtaining at least one biological sample from the animal, and/or recording clinical parameters of the animal; and, where at least one biological sample has been obtained,
(iv) analysing the biological sample for at least one analyte.

The term ‘preclinical drug studies’ will be understood to include any drug study performed in an animal before or in addition to human drug trials. Suitably, in some embodiments, preclinical drug studies may refer to toxicity studies, drug to drug interaction studies, pharmacokinetic studies, pharmacodynamic studies and mechanistic studies.

In some embodiments the method may comprise obtaining a second, a third, a fourth, a fifth, a sixth, a seventh, an eighth, a ninth or a tenth biological sample. In a preferred embodiment, a plurality of biological samples are obtained. Accordingly, steps (iii) and (iv) may be performed at more than one time point. In one embodiment, steps (iii) and (iv) are performed a plurality of times at more than one time point.

Suitably, in some embodiments, the at least one analyte of the method may be a drug metabolite, a drug metabolising enzyme, a liver enzyme, a gene, RNA, a protein, a transcription factor or a cell.

In an another aspect, there is provided a method of measuring rate of metabolism of a drug compound, wherein the method comprises:
(i) providing an immunodeficient non-human animal of the first or fourth aspect;
(ii) administering at least one test compound to the animal;
(iii) obtaining at least one biological sample from the animal, and/or recording clinical parameters of the animal; and, where at least one biological sample has been obtained,
(iv) measuring the rate of metabolism, e.g. the half-life, of the compound in the biological sample.

Suitably, in some embodiments the half-life of the compound is measured by measuring toxicity to the animal, measuring activity of the compound, measuring the level of a transcription factor or drug metabolising enzyme.

In the eighth or ninth aspects as described herein, the test compounds may be administered by topical or systemic administration. Systemic administration may be enteral (e.g. oral, sublingual, and rectal) or parenteral (e.g. injection). Suitable methods of administration may be enteral (e.g. oral, sublingual, and rectal) or parenteral (e.g. injection) including intravenous, intraarterial, intracranial, intramuscular, subcutaneous, intra-articular, intrathecal, and intradermal injections. Preferred administration methods are intravenous, intraarterial, intracranial and intrathecal injection. In some embodiments where more than one test compound is being administered, the test compounds may be administered concurrently or sequentially. In some embodiments, the test compound may be administered to the immunodeficient non-human animal in a suitable carrier.

For example, in some embodiments the rate of metabolism of the compound may be measured as the rate of formation of the oxidized product or the formation of a subsequent product generated from the oxidised intermediate. Alternatively, the rate of metabolism may be represented as the half-life or rate of disappearance of the initial compound or as the change in toxicity or activity of the initial compound or a metabolite generated from the initial compound. The half-life may be measured by determining the amount of the drug compound present in samples taken at various time points. Suitably, in some embodiments the method may comprise obtaining a second, a third, a fourth, a fifth, a sixth, a seventh, an eighth, a ninth or a tenth biological sample. In a preferred embodiment, a plurality of biological samples are obtained. Accordingly, steps (iii) and (iv) may be performed at more than one time point. In one embodiment, steps (iii) and (iv) are performed a plurality of times at more than one time point. The amount of the drug compound may be quantified using standard methods such as high-performance liquid chromatography, mass spectrometry, western blot analysis using compound specific antibodies, or any other appropriate method.

It is also possible in some embodiments to examine whether under particular circumstances a test compound is metabolised to a toxic or carcinogenic metabolite, for example, by measuring its covalent binding to tissues, proteins or DNA or by measuring glutathione depletion.

According to the methods as described herein, in some embodiments, the method may comprise a further step of transplanting human cells into the immunodeficient non-human animal prior to, simultaneously to, or immediately after performing step (ii) of the method. Suitably, in some embodiments the human cells are patient-derived cells or healthy donor cells. Healthy donor cells may include cells from a healthy, non-diseased individual or cells isolated from the same subject as the patient-derived cells but those unaffected by the condition or disease. In some embodiments, the human cells are cell-line derived or primary human cells.

In some embodiments of the methods described herein, the biological sample may be any one selected from the list comprising: blood, plasma, serum, urine, stool, hair, skin, bone marrow, tissue or saliva. In some embodiments, the non-human animal may be sacrificed in step (iii) of the method to obtain a biological sample. Such biological samples obtained at sacrifice may include any organ or tissue for example, but not limited to, heart, lung, liver, kidney, spleen, brain, skin, bones, bone marrow, pancreas, eyes, reproductive organs, thymus, gall bladder, stomach and intestines. It may be desirable in some embodiments to recover the human cells following administration of the test compound. Suitably, in some embodiments, a further step (v) may be performed, wherein the transplanted human cells may be recovered from the biological sample obtained in step (iii) of the method.

In embodiments wherein the transplanted human cells are derived from a diseased patient, the immunodeficient non-human animal may be considered a disease-specific model. In some embodiments, the disease specific model is a model of any one of cancer, infectious disease and inflammatory disease.

In some embodiments, the method comprises xenotransplantation of cancer cell lines, patient-derived primary cancer cells, metastatic cancer cells, cancer associated stromal cells (e.g. cancer-associated fibroblasts and mesenchymal cells), and cancer stem cells. Suitably, it will be apparent to the skilled person that healthy cells include non-transformed cells derived from a patient, or suitable healthy control cells can also be transplanted into the immunodeficient animal for use in the methods described herein.

Suitably, in some embodiments, patient-derived cancer cells can be derived from any cancer type. Suitably, cancer types may include, but are not limited to, any of melanoma, neuroblastoma, colorectal cancer, bowel cancer, breast cancer, pancreatic cancer, leukaemia, multiple myeloma, hepatocellular carcinoma, lymphoma, bone cancer, throat cancer, oesophageal cancer, skin cancer, liver cancer, kidney cancer, bladder cancer, ovarian cancer, cervical cancer, brain cancer, colon cancer, thyroid cancer and lung cancer.

In one aspect, there is provided herein a method of testing at least one pharmaceutical compound, wherein the method comprises:
(i) providing an immunodeficient non-human animal according to any one of the first or fourth aspects;
(ii) administering at least one test compound to the animal; and
(iii) obtaining at least one biological sample from the animal, and/or recording clinical parameters of the animal; and optionally where at least one biological sample has been obtained,
(iv) analysing the biological sample for at least one analyte or clinical parameter.

Suitably, in a further aspect, there is provided herein a method of testing anti-cancer compounds, wherein the method comprises:
(i) providing an immunodeficient non-human animal according to any one of the first or fourth aspects;
(ii) transplanting healthy and/or cancerous human cells into the animal;
(iii) obtaining at least one biological sample from the animal, and/or recording clinical parameters of the animal; and, where at least one biological sample has been obtained,
(iv) analysing the biological sample for at least one analyte or clinical parameter.

Clinical parameters as referred to in any embodiment herein, may refer to, but are not limited to any one or more physiological measurements of the animal such as weight, heart rate, activity levels, appetite, tumour burden, tumour size, tumour weight, light sensitivity, hunching, hair loss and lethargy.

Disease-specific models may also include infectious disease models. Infectious disease models may include, but are not limited to, human immunodeficiency virus (HIV), SARS-COV-2 (Covid-19), hepatitis A, hepatitis B, hepatitis C, parasitic diseases such as malaria, toxoplasmosis, chagas disease, African sleeping sickness, hydatid disease, hook worm infections, round worm infections and leishmania.

Currently, no small animal model is capable of maintaining the complete life cycle of Plasmodium parasites that infect humans in malaria infections. As a result, developing and testing anti-malarial drugs is challenging.

Suitably, in a further aspect, there is provided a method of testing an anti-malarial compound, wherein the method comprises:
(i) providing an immunodeficient non-human animal of the first or fourth aspect;
(ii) infecting the animal with a Plasmodium parasite;
(iii) obtaining at least one biological sample from the animal, and/or recording clinical parameters of the animal; and, where at least one biological sample has been obtained,
(iv) analysing the biological sample for at least one analyte or clinical parameter.

Suitably, in some embodiments, the plasmodium parasite is any one of Plasmodium falciparum, Plasmodium vivax, Plasmodium ovale, Plasmodium malariae, or Plasmodium knowlesi.

In one embodiment, the method may comprise a further step of transplanting human cells into the immunodeficient non-human animal prior to, or simultaneously to, or immediately after, performing step (ii) of the method.

Step (iv) of the method, may comprise analysing the number of infected human cells, measuring a drug metabolite, measuring a drug metabolising enzyme, measuring a liver enzyme, measuring a gene, measuring RNA, measuring a protein, measuring a transcription factor or measuring activity of the at least one compound.

### BRIEF DESCRIPTION OF THE FIGURES

FIG. 1: A. Xenograft of BRAFV600E human melanoma A375 cells in Rag2^{null}/8HUM mice (top). Bottom-no growth of A375 cells in immunocompetent 8HUM mice. B. Growth of a murine melanoma syngeneic graft in 8HUM mice and response to dabrafenib treatment. Adult female 8HUM_Rag2−/− mice (18-21w, n=5) were injected subcutaneously (s.c.) in one flank with 3.5×106 5555 murine melanoma cells, in 100 μl ECM diluted 1:1 with DMEM. Tumours were allowed to establish and on day 5 after implantation daily treatment was commenced with either vehicle (0.5% (w/v) hydroxypropylmethylcellulose, 0.2% (v/v) Tween 80; closed circles) or dabrafenib methanesulfonate (in vehicle, open circles) suspended at 6.3 mg/ml and administered at 5 ml/kg, such that dabrafenib dose administered was 31.5 mg/kg (arrow). Tumour measurements were taken three times weekly, then daily as required, and tumour volume calculated as detailed in Methods section. The study was terminated 15 days after implantation of cells. Data shown are mean tumour volume±SEM.

FIG. 2: Response of A375 human melanoma xenograft to dabrafenib treatment in 8HUM_Rag2^{-/-} mice. Adult female 8HUM mice (11-19w, n=3) were injected s.c. in both flanks with 4.4×10⁶ A375 melanoma cells, in 100 μl DMEM. Tumours were allowed to establish and on day 28 after implantation daily treatment was commenced with either vehicle (0.5% (w/v) hydroxypropylmethylcellulose, 0.2% (v/v) Tween-80; closed circles) or dabrafenib (in vehicle, open circles) suspended at 6.3 mg/ml and administered at 5 ml/kg, such that dabrafenib dose administered was 31.5 mg/kg (arrow). Tumour measurements were taken three times weekly, then daily as required, and total volume of tumours on both flanks was calculated as detailed in Methods section. The study was terminated on d35 after implantation of cells, although one vehicle-treated animal had to be removed from the study on d29 as its total tumour size approached the maximum permitted under legislation.

Data shown are mean tumour volume±SEM.

FIG. 3: Response of A375 human melanoma xenograft to trametinib treatment in 8HUM/Rag2^{-/-} mice. Adult female 8HUM mice (8-18w, n=3 or 4) were injected s.c. in one flank with 5×106 A375 melanoma cells, in 100 μl DMEM. Tumours were allowed to establish and on day 22 after implantation daily treatment was commenced with either vehicle (0.5% (w/v) hydroxypropylmethylcellulose, 0.2% (v/v) Tween 80; closed circles) or trametinib (in vehicle, open circles) suspended at 0.07 mg/ml and administered at 5 ml/kg, such that trametinib dose administered was 0.35 mg/kg (arrow). Tumour measurements were taken three times weekly, then daily as required, and tumour volume calculated as detailed in Methods section. Vehicle-treated mice were sacrificed on d30 after implantation of cells; trametinib treatment was discontinued (‘No’ symbol) at that time for the drug-treated group to determine whether tumour re-growth would occur in the absence of drug. Data shown are mean tumour volume±SEM. FIG. 4: CYP1A1/1A2/Cyp1a. Strategy to generate hCYP1A1/1A2 and Cyp1a KO mice. A. Genomic organisation of the mouse Cyp1a1/1a2 gene locus. The start ATGs and stop codons are shown. B. Genomic organisation of Cyp1a1/1a2 in targeted ES cells after homologous recombination. C. Cyp1a1/1a2 gene locus in the hCYP1A1/1A2 model after Flp-mediated deletion of the neomycin (NeoR) and puromycin (PuroR) expression cassettes. D. Cyp1a1/1a2 gene locus in the Cyp1a KO model after Cre-mediated deletion. For the sake of clarity sequences of the targeting vectors are not drawn to scale. pA=polyadenylation signal, hGHpA=polyadenylation signal of human growth hormone. Kapelyukh et al., Drug Metab. Disp. (2019) 47, 907 PMID: 31147315.

FIG. 5: CYP2C9/Cyp2c. Strategy for generating Cyp2c KO and hCYP2C9 mice A. schematic representation of the chromosomal organization and orientation of functional genes within the mouse Cyp2c cluster. B. exon/intron structure of Cyp2c55 and Cyp2c70. Exons are represented as black bars, and the ATGs mark the translational start sites of both genes. The positions of the targeting arms for homologous recombination are highlighted in grey (Cyp2c55) and black (Cyp2c70), respectively. C. vectors used for targeting of Cyp2c55 (left) and Cyp2c70 (right) by homologous recombination. loxP, lox5171, frt, and f3 sites are represented as white, stripped, black, and grey triangles, respectively. D. genomic organization of the Cyp2c cluster in double-targeted ES cells after insertion of the targeting vectors. E. deletion of the mouse Cyp2c cluster after Cre-mediated recombination at the loxP sites. F. CYP2C9 expression cassette used for Cre-mediated insertion via the loxP and lox5171sites. G. mouse Cyp2c locus after Cre-mediated insertion of the CYP2C9 expression cassette. H. mouse Cyp2c locus in the hCYP2C9 model after Flp-mediated deletion of the neomycin expression cassette. For the sake of clarity sequences are not drawn to scale. Hyg, hygromycin expression cassette; TK, thymidine kinase expression cassette; alb Prom, mouse albumin enhancer/promoter element; P, promoter that drives the expression of neomycin; 5′ΔNeo, ATG-deficient neomycin. Scheer et al., Mol. Pharmacol. (2012) 82, 1022 PMID: 22918969.

FIG. 6: CYP2D6/Cyp2d. Strategy for the deletion of the mouse Cyp2d cluster and insertion of human CYP2D6 expression cassettes. A. schematic representation of the chromosomal organization and orientation of functional genes within the mouse Cyp2d cluster. B. exon/intron structure of Cyp2d22 and Cyp2d26. Exons are represented as black bars and the ATGs mark the translational start sites of both genes. The positions of the targeting arms for homologous recombination are highlighted in light (Cyp2d22) and dark grey (Cyp2d26), respectively. C. vectors used for targeting of Cyp2d22 (left) and Cyp2d26 (right) by homologous recombination. loxP and frt sites are represented as white and black triangles, respectively. CYP2D6 expression cassettes consisting of a 9-kb promoter sequence (dotted bar) and all exons, introns, and 5′ and 3′ untranslated regions (dotted arrow) are included in the Cyp2d22 targeting vector. D. genomic organization of the Cyp2d cluster in double-targeted ES cells after insertion of the targeting vectors. E. deletion of the mouse Cyp2d cluster after Cre-mediated recombination at the loxP sites. F. knockout allele of the Cyp2d cluster after Flp-mediated deletion of the CYP2D6 expression cassette. For the sake of clarity sequences are not drawn to scale. Hyg, hygromycin expression cassette; Neo, neomycin expression cassette; ZsGreen, ZsGreen expression cassette. Scheer et al., Mol. Pharmacol. (2012) 81, 63 PMID: 21989258.

FIG. 7: CYP3A4/7/Cyp3a. Strategy to generate Cyp3a(−/−)/Cyp3a13(+/+) and huCYP3A4/3A7 mice. A. schematic representation of the chromosomal organization and orientation of functional genes within the mouse Cyp3a cluster. Pseudogenes are not listed. B. exon/intron structure of Cyp3a57 and Cyp3a59. Exons are represented as black bars, and the ATGs mark the translational start sites of both genes. The positions of the targeting arms for homologous recombination are highlighted as light (Cyp3a57) and dark (Cyp3a59) grey lines, respectively. C. vectors used for targeting of Cyp3a57 (left) and Cyp3a59 (right) by homologous recombination. D. genomic organization of the Cyp3a cluster in double-targeted ES cells after homologous recombination on the same allele at the Cyp3a57 and Cyp3a59 locus. E. deletion of the mouse Cyp3a cluster after Cre-mediated recombination at the loxP sites. F. modified human BAC containing CYP3A4 and CYP3A7 used for Cre-mediated insertion into the loxP and lox5171 sites of the prepared Cyp3a knockout locus. G. targeted mouse Cyp3a locus after Cre-mediated recombination of the modified human BAC into the loxP and lox5171 sites of the Cyp3a knockout allele. H. humanized Cyp3a locus after Flp-mediated deletion of the frt and f3-flanked hygromycin and neomycin expression cassettes. The ES cells shown in E and H were used to generate the Cyp3a(−/−)/Cyp3a13 (+/+) and huCYP3A4/3A7 mice, respectively, as described under Materials and Methods. For the sake of clarity, sequences are not drawn to scale. LoxP, lox5171, frt, and f3 sites are represented as white, striped, black, or grey triangles, respectively. TK, thymidine kinase expression cassette; Hygro, hygromycin expression cassette; ZsGreen, ZsGreen expression cassette; P, =promoter that drives the expression of neomycin; 5′Δ Neo, ATG-deficient Neomycin. Hasegawa et al., Mol. Pharmacol. (2011) 80, 518 PMID: 21628639.

FIG. 8: CAR/PXR/Car/Pxr. Strategy to generate huPXR, huCAR, and KO mice. A. human PXR minigene, containing a fusion of exons 2 through 4, genomic sequences between exons 4 and 8, and a fusion of exons 8 and 9, was knocked in onto the translational start ATG of the mouse PxrWT gene to generate PXR-targeted mice. Mouse exons are indicated in black vertical lines and with small type. Human exons are white boxes with capitals, and the targeting arms are shown as dark and light grey lines. Targeted mice were crossed to a mouse strain expressing the FLPe recombinase to delete the hygromycin selection cassette by FLP-mediated recombination at the FRT sites (white triangles) and to generate huPXR mice. For the sake of clarity, sequences of the targeting vector are not drawn to scale. Scheer et al., Drug Metab. Disp. (2010) 38, 1046 PMID: 20354104. B. CAR: The coding region of the mCar WT gene was replaced with the genomic coding region of hCAR, including exons 2-9, in order to generate CAR-targeted mice. In both cases, mouse exons are indicated in black and with lower-case letters; human exons are indicated in white and with upper-case letters. Targeted mice are crossed to a mouse strain expressing the FLPe recombinase to delete the hygromycin or neomycin selection cassette, respectively, and to generate huPXR or huCAR mice or to a φC31 deleter strain to generate PXR or CAR KO mice. For the sake of clarity, sequences of the targeting vector are not drawn to scale. Scheer et al., J. Clin. Invest. (2008) 118, 3228 PMID: 18677425.

FIG. 9: 8HUM Rag2^{-/-} targeting strategy. The targeting strategy is based on NCBI transcript NM_009020.3 which corresponds to Ensembl transcript ENSMUST00000044031. Rag2 targeted coding region indicated. Proximal guide RNA (gRNA1) and distal guide RNA (gRNA2); see sequences below.

FIG. 10: 8HUM_IL2Rγ^{-/-} targeting strategy. The targeting strategy is based on NCBI transcript NM_013563.4 which corresponds to Ensembl transcript ENSMUST00000033664.13. IL2Rγ targeted coding region indicated. Proximal guide RNA (gRNA1) and distal guide RNA (gRNA2); see sequences below.

FIG. 11: Mortality rates of male and female mice. Genotype 8HUM: Rag2: Il2Rγ. Number of mice found dead from genetic backgrounds Hum KO null (8Hum Rag2^{-/-} IL2Rγ^{-/-}); Hum KO Het (8Hum Rag2^{-/-} IL2Rγ^{+/-}); Hum KO WT (8Hum Rag2^{-/-} IL2Rγ^{+/+}); Het KO null (8Hum^{CYP2C9+/-} Rag2^{-/-} IL2Rγ^{-/-}); Het KO Het (8Hum^{CYP2C9+/-} Rag2^{-/-} IL2Rγ^{+/-}); Het KO WT (8Hum^{CYP2C9+/-} Rag2^{-/-} IL2Rγ^{+/+}); Het WT Null (8Hum^{CYP2C9+/-} Rag2^{+/+} IL2Rγ^{-/-}); Hum WT Null (8Hum Rag2^{+/+}IL2Rγ^{-/-}); Hum Het Null (8Hum Rag2^{+/-} IL2Rγ^{-/-}); Het Het Het (8Hum^{CYP2C9+/-} Rag2+/−IL2Rγ^{+/-}); unknown (not genotyped).

### DETAILED DESCRIPTION OF EMBODIMENTS OF THE INVENTION AND EXAMPLES

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not limit the scope of the invention.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Ausubel, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); U.S. Pat. No. 4,683,195; Nucleic Acid Hybridization (Harries and Higgins eds. 1984); Transcription and Translation (Hames and Higgins eds. 1984); Culture of Animal Cells (Freshney, Alan R. Liss, Inc., 1987); Immobilized Cells and Enzymes (IRL Press, 1986); Perbal, A Practical Guide to Molecular Cloning (1984); the series, Methods in Enzymology (Abelson and Simon, eds.-in-chief, Academic Press, Inc., New York), specifically, Vols. 154 and 155 (Wu et al. eds.) and Vol. 185, “Gene Expression Technology” (Goeddel, ed.); Gene Transfer Vectors For Mammalian Cells (Miller and Calos eds., 1987, Cold Spring Harbor Laboratory); Immunochemical Methods in Cell and Molecular Biology (Mayer and Walker, eds., Academic Press, London, 1987); Handbook of Experimental Immunology, Vols. I-IV (Weir and Blackwell, eds., 1986); and Manipulating the Mouse Embryo, (Cold Spring Harbor Laboratory Press, Cold Spring Harbor, N.Y., 1986).

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

### Introduction

Although the use of genetically modified animals poses questions of an ethical nature, the benefit to man from studies of the types described herein is considered vastly to outweigh any suffering that might be imposed in the creation and testing of genetically modified animals. As will be evident to those of skill in the art, drug therapies require animal testing before clinical trials can commence in humans under current regulations. With currently available model systems, animal testing cannot be dispensed with. Whenever a drug fails at a late stage in testing, all of the animal experiments will in a sense have been wasted. Stopping drugs failing therefore saves test animals' lives. Therefore, although the present invention relates to genetically modified animals, the use of such animals should reduce the number of animals that must be used in drug testing programmes.

The inventors of the present immunodeficient animal model have provided a potential solution to reducing the drug development attrition rate and therefore potentially reducing the total number of animals needed for animal studies. However, the immunodeficient animal model of the present invention was not developed without challenge. As discussed above, the immunodeficient animal model can comprise a remarkable 35 gene inactivation, 8 gene loci humanization on an immunodeficient background. Remarkably, the inventors have developed this animal model without significant early life mortality or severe and undesirable phenotypes therefore the model as described herein has no or minimal suffering to the animal.

As will be apparent to the skilled person, the immunodeficient non-human animal of the present invention provides a new opportunity to study drug metabolism in the presence of patient-derived cells which may provide a substantial benefit in improving the success rate of clinical trials and the translation of drugs from the bench to the clinic.

### Definitions

By the term “substantially inactive” as used herein it is meant that the endogenous drug metabolism enzyme and endogenous transcription factor has less or no function when compared to the native protein. It is preferred that the endogenous drug metabolism enzyme and endogenous transcription factor when substantially inactivate, is at least 50%, at least 60%, at least 70%, at least 80%, at least 90%, at least 95%, at least 96%, at least 97%, at least 98%, at least 99% or 100% less functional than the native protein. Genes encoding the endogenous metabolism enzyme or endogenous transcription factor are considered substantially inactivated when the endogenous gene is unable to express the gene product(s), at least not to any level that is significant to the drug metabolism process. For instance, the expression level of a substantially inactivated gene may be less than 20%, preferably less than 10%, more preferably less than 5%, more preferably less than 2%, even more preferably 1% or less of the wild type expression level. The expression of a substantially inactivated gene may preferably be decreased to the point at which it cannot be detected.

The drug metabolism system as described herein includes P450 drug metabolising enzymes and any other protein that is involved in xenobiotic metabolism. The skilled person would understand that it may be desirable to substantially inactivate any endogenous protein involved in drug metabolism and to further introduce any human gene(s) encoding proteins involved in drug metabolism. For example, all cytochrome P450s receive electrons from a single donor, cytochrome P450 reductase (CPR) and deletion of this protein would therefore inactivate all P450-mediated metabolism. While complete deletion of CPR is lethal at the embryonic stage of development, mice where the CPR gene is flanked with loxP sequences so that CPR can be conditionally deleted in the postnatal period in a specific tissue by developmentally controlled expression of Cre recombinase can survive to adulthood in good health. For instance, it is known to produce and use a Hepatic Reductase Null (“HRNT”) mouse in which the CPR enzyme on which all P450s depend has been deleted in the liver. HRNTM mice therefore completely lack P450-mediated metabolism in the liver. It is within the scope of the present invention to introduce human drug metabolism enzymes into a HRN™ mouse and render said mouse immunodeficient through substantial inactivation of endogenous Rag2 or Prkdc.

An immunodeficient model as referred to herein may refer to any non-human animal model that lacks a functional component of the immune system. The animal may be deficient in any part of the innate or, more preferably, the adaptive arm of the immune system. This immunodeficiency can arise as a result of genetic manipulation, chemical inhibition, irradiation, surgical methods such as thymectomised animals or any combination thereof. An immunodeficient model in the context of the present invention includes any model that permits xenograft transplantation. An immunodeficient model may lack functional T, B and/or natural killer (NK) cells. An immunodeficient mouse may have a Rag2 deletion. A Rag2^{null} animal has no functional mature T or B cells. An immunodeficient animal may also include a NOD SCID animal, where a homozygous SCID mutation on a non-obese diabetic (NOD) background results in a failure to develop functioning T, B and NK cells.

The term “vector” is well known in the art, and as used herein refers to a nucleic acid molecule, e.g. double-stranded DNA, which may have inserted into it a nucleic acid sequence according to the present invention. A vector is suitably used to transport an inserted nucleic acid molecule into a suitable host cell. A vector typically contains all of the necessary elements that permit transcribing the insert nucleic acid molecule, and, preferably, translating the transcript into a polypeptide. A vector typically contains all of the necessary elements such that, once the vector is in a host cell, the vector can replicate independently of, or coincidental with, the host chromosomal DNA; several copies of the vector and its inserted nucleic acid molecule may be generated. Vectors of the present invention can be vectors that integrate into the host cell genome. This definition includes both non-viral and viral vectors. Non-viral vectors include but are not limited to plasmid vectors (e.g. pMA-RQ, pUC vectors, bluescript vectors (pBS) and pBR322 or derivatives thereof that are devoid of bacterial sequences (minicircles)) transposons-based vectors (e.g. PiggyBac (PB) vectors or Sleeping Beauty (SB) vectors), etc. Larger vectors such as artificial chromosomes (bacteria (BAC), yeast (YAC), or human (HAC)) may be used to accommodate larger inserts. Viral vectors are derived from viruses and include but are not limited to retroviral, lentiviral, adeno-associated viral, adenoviral, herpes viral, hepatitis viral vectors or the like. Typically, but not necessarily, viral vectors are replication-deficient as they have lost the ability to propagate in a given cell since viral genes essential for replication have been eliminated from the viral vector. However, some viral vectors can also be adapted to replicate specifically in a given cell, such as e.g. a cancer cell, and are typically used to trigger the (cancer) cell-specific (onco) lysis. Virosomes are a non-limiting example of a vector that comprises both viral and non-viral elements, in particular they combine liposomes with an inactivated HIV or influenza virus (Yamada et al., 2003). Another example encompasses viral vectors mixed with cationic lipids.

A “functional variant” of a nucleic acid construct in the context of the present invention is a variant of a reference sequence that retains the ability to function in the same way as the reference sequence. Alternative terms for such functional variants include “biological equivalents” or “equivalents” or “allelic variants”.

The term “orthologue” as used herein refers to genes of different species which evolved from a common ancestral gene that typically have retained a similar function in different species.

The term “homologue” as used herein refers to a gene inherited in two species by a common ancestor.

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215:403-10), such as the “Blast 2 sequences” algorithm described by Tatusova and Madden 1999 (FEMS Microbiol Lett 174:247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

“Toxicity studies” as referred to herein relates to investigations that determine the safety profile of candidate drugs. Toxicity studies include determining drug absorption, distribution, metabolism, and excretion of the drug in a model organism. A toxicity study may include any toxicology study that may be necessary to include in a non-clinical programme, such as single-dose studies, repeated-dose studies, reproductive toxicology studies, local toxicology studies e.g. tolerance of skins and eyes to compound, hypersensitivity studies, genotoxicity studies and carcinogenicity studies.

Pharmacokinetics (PK) describes how the body affects a specific drug after administration through the mechanisms of absorption and distribution, as well as the metabolic changes of the substance in the body and the effects and routes of excretion of the metabolites of the drug. PK properties of chemicals are affected by the route of administration and the dose of administered drug, which may affect absorption rate. PK includes investigating the process of release of a drug from the pharmaceutical formulation, the process of a substance entering the blood circulation, the dispersion or dissemination of substances throughout the fluids and tissues of the body, the recognition by the organism that a foreign substance is present and the irreversible transformation of parent compounds into daughter metabolites and the removal of the substances from the body. In rare cases, some drugs irreversibly accumulate in body tissue.

Pharmacodynamics (PD) describes the study of the biochemical and physiologic effects of drugs on the body. PD includes assessing the desired effects such as enzyme inhibition, assessing off-target side effects, determining the therapeutic window between the effective dose and undesirable side effects and determining the half-life of drugs.

Drug to drug interaction studies include any study where the effect of more than one drug in combination is investigated. Drug to drug interaction studies can include toxicity, PK and PD studies, as well as assessment of drug synergy, antagonism and staggered dosing regimens.

Mechanistic studies as described herein include any study designed to understand a biological or behavioural process, the pathophysiology of a disease, or the mechanism of action of an intervention.

The term “efficacy” as used herein describes the maximum response that can be achieved with a drug. A dose-response curve, where the effect of the drug is plotted against dose in a graph can be used to determine the maximum response (Emax). The highest point on the curve shows the maximum efficacy.

The term “xenograft” as used herein refers to a cell, tissue or organ that is derived from a species that is different from the recipient of the cell, tissue or organ. In the context of the present invention, xenografts or xenotransplantation refers to the transplantation of human cells into the immunodeficient non-human animal. Patient-derived xenografts refer to transplantation of tissue or cells from a patient into an immunodeficient transgenic animal. Cell line derived xenografts refer to the transplantation of immortalised cell lines into an immunodeficient transgenic animal.

The term ‘homozygous’ refers to having two identical alleles of a particular gene or genes. The term ‘heterozygous’ refers to having two different alleles of a particular gene or genes.

The term “analyte” as referred to herein may refer to any substance of interest. An analyte may include, but is not limited to, a metabolite, preferably a drug metabolite; an enzyme, preferably a drug metabolising enzyme or a liver enzyme; a gene; RNA (e.g. mRNA, tRNA, rRNA); a protein; a transcription factor; pH; or a cell.

Test compound as referred to herein may refer to any chemical or biological compound being investigated. Examples of test compounds include, but are not limited to, small molecule inhibitors, biologics (e.g. monoclonal antibodies, mono- and bi-valent antibody fragments, monobodies, nanobodies), antibiotics, probiotics, viral agents, gene therapy vectors, vaccines nucleic acid based drugs, or cells (e.g. CAR-T cells, stem cells, NK cells, CD8+ T cells).

Treating a cell in vitro or ex vivo as used herein may refer to providing a test compound to a cell that is cultured or isolated directly from an animal of the invention. It will be understood by the skilled person a test compound may be provided to a cell in a suitable carrier solvent.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. Suitable carriers may be readily selected by one of skill in the art in view of the indication for which the pharmaceutical composition is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure. Various other conventional pharmaceutical ingredients may be provided in the pharmaceutical composition, such as preservatives or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

Delivery vehicles such as liposomes, nanocapsules, microparticles, microspheres, lipid particles, vesicles, and the like, may be used for the introduction of test compounds to suitable cells or animals of the invention or to deliver transgenes to the animal of the invention. In particular, the vector delivered transgenes may be formulated for delivery either encapsulated in a lipid particle, a liposome, a vesicle, a nanosphere, or a nanoparticle or the like.

Embodiments of the present invention will now be described, by way of non-limiting example, with reference to the accompanying drawings.

### EXAMPLES

### Introduction

Drug development is an expensive and time-consuming process, with only a small fraction of drugs gaining regulatory approval from the often many thousands of candidates identified during target validation. Once a lead compound has been identified and optimised, they are subject to intensive pre-clinical research to determine their pharmacodynamic, pharmacokinetic and toxicological properties, procedures which inevitably involve significant numbers of animals-mainly mice and rats, but also dogs and monkeys in much smaller numbers and for specific types of drug candidates. Many compounds that emerge from this process, having been shown to be safe and efficacious in pre-clinical studies, subsequently fail to replicate this outcome in clinical trials, therefore wasting time, money and, most importantly, animals.

The poor predictive power of animal models in pre-clinical studies is predominantly due to lack of efficacy or safety reasons, which in turn can be attributed mainly to the significant species differences in drug metabolism between humans and animals. To circumvent this, we have developed a complex transgenic mouse model-8HUM-which faithfully replicates human Phase I drug metabolism (and its regulation), and which will generate more human-relevant data [REFINEMENT] from fewer animals [REDUCTION] in a pre-clinical setting and reduce attrition in the clinic. One key area for the pre-clinical application of animals in an oncology setting—almost exclusively mice—is their use in anti-tumour studies. We now further demonstrate the utility of the 8HUM mouse using a murine melanoma cell line as a syngeneic tumour and also present an immunodeficient version 8HUM_Rag2^{-/-} for use in xenograft studies. These models will be of significant benefit not only to Pharma for pre-clinical drug development work, but also throughout the drug efficacy, toxicology, pharmacology, and drug metabolism communities, where fewer animals will be needed to generate more human-relevant data.

The pre-clinical stage of drug development provides crucial information for the decision process as to whether a drug candidate will proceed to ‘first in human’ and phased clinical trials (7). The failure rate through the pre-clinical stages of drug development can be high and many candidate molecules taken forward to clinical trials subsequently fail to recapitulate the safety profile and efficacy found in animal studies (8-11). There are many reasons for this, but significant species differences in drug metabolism between animals (rats, mice) and humans—with concomitant changes in pharmacokinetics, metabolite profiles, toxicokinetics and pharmacodynamics—are key components underlying the observed failure rates (11). We have developed a sophisticated transgenic model in which the major human drug metabolising enzymes—and the transcription factors regulating their expression-replace their mouse counterparts. In a previous report on this humanised mouse model (12) we show, using model compounds and anti-cancer drugs, that drug metabolism and disposition in the 8HUM mouse more closely reflects that found in humans. Given the growing importance of drug combinations in cancer therapy (13), it is clear that a genetically engineered mouse model such as 8HUM could play a pivotal role in the development of such combinations (14).

While some pre-clinical work is carried out in vitro, using a variety of cell lines including immortalised human cells, much—and arguably the most important—is carried out in animals, mainly rodents but also dogs, and for certain types of candidate molecules, primates. One such in vivo use in the pre-clinical setting is syngeneic or xenograft work, where anti-tumour efficacy of drug candidates is tested alone or in drug combinations. In a syngeneic model, murine cell lines are implanted subcutaneously or orthotopically and tumour response to candidate drugs tested. Whilst such experiments can account for the effect of immune system, the genetic background of the tumour cells—which must match that of the host animal used—can also give rise to disparate results. More recently xenograft models have come to the fore, where immunodeficient mouse lines are able to grow human tumours either from existing immortalised cell lines or via fresh tissue as patient-derived xenografts (PDX) (15, 16). Despite lack of a competent immune system and any issues that this may potentially cause in the interpretation of results, the latter are growing in use, a good example being the EurOPDX consortium, who have a database of PDX models to share with the research community (17). Notwithstanding extensive xenograft use in various guises, such models still have issues arising from retention of murine drug metabolism and disposition (14).

Examples shown herein demonstrate a modification of the humanised 8HUM model in which the inventors have generated a compromised immune system by deleting the Rag2 locus. Using murine and human melanoma cell lines in 8HUM_Rag2^{-/-} mice, shown is tumour growth in a syngeneic and xenograft setting, respectively, and demonstrate in vivo sensitivity to dabrafenib and trametinib, drugs currently used in combination as standard of care in the treatment of metastatic melanoma. Together, these models have the potential to reduce the number of animals used in the pre-clinical stages of drug development, while potentially generating more human-relevant data and thus potentially improving the chances of a candidate drug replicating a positive pre-clinical finding in successful clinical trials.

### Methods

### Reagents

Unless specifically stated, reagents used in these studies were purchased from Sigma-Aldrich (Dorset, UK).

### Animals

### Generation of 8HUM Mice

Transgenic mice-8HUM—extensively humanized for the major cytochrome P450 enzymes in Phase I drug metabolism, along with the transcription factors regulating their expression, have previously been described by Henderson et al. (12) and were generated in a collaboration between CXR Biosciences and Taconic Biosciences funded through the Scottish Government ITI, with CRW as one of the principal investigators.

Thirty-five murine genes (the Cyp2c (except Cyp2c44), Cyp2d, and Cyp3a murine gene clusters and transcription factors Car and Pxr) were replaced by eight human genes (CYP1A1, CYP1A2, CYP2C9, CYP2D6, CYP3A4, CYP3A7, CAR, PXR). Expression of human P450 genes was from the human promotor, except for CYP2C9, which was driven by the albumin promotor, and CYP1A1, CYP1A2, CAR and PXR which were driven off the corresponding murine promoters.

The 8HUM mouse was created by inter-crossing a series of mouse lines in which specific mouse Cyp gene clusters had been deleted and replaced with the corresponding human gene(s) driven either by the human promotor, or the mouse promotor, or in some instances by the albumin promotor. In addition, mouse lines had been generated in which the two major transcription factors responsible for regulating the expression of P450 genes—the constitutive androstane receptor (Car) and the pregnane X reporter (Pxr)—were deleted and replaced with the human orthologues (CAR, PXR). References to published material reporting these mouse lines are also listed below by PubMed number (8Hum mouse generated as described in Henderson et al., Drug Metab. Disp. (2019) 47, 601 PMID: 30910785).

The final mouse line is designated thus:
hP_hC_h3A4_3A7_8Cyp3aKO_albCYP2C9_h2D6.2_h1A1_1A2 HU ITI0153
‘8HUM’
C57BL/6-Nr1i2^{tm1198(NR112)Arte}-Nr1i3^{tm1089(NR113)Arte}-Cyp3a^{tm1109(CYP3A4/3A7)Arte}-Cyp3a13^{tm1728Arte}-Cyp2d^{tm1836(CYP2D6*2)Arte}-Cyp2c^{tm1897(albCYP2C9)Arte}-CYP1a1^{tm2025(CYP1A1)Arte}-Cyp1a2^{tm2024(CYP1A2)Arte}

The 8HUM line was maintained as ‘heterozygous’ at the CYP2C9/Cyp2c locus—i.e. one allele is the mouse Cyp2c cluster, the other is deleted for Cyp2c and humanised for CYP2C9. It was found empirically that this is required to optimise fecundity though the precise reason for this is not yet known.

### Generation of 8HUM Rag2^{-/-} Mice

8HUM mice were further genetically altered by deleting Rag2 using CRISPR/cas9-mediated gene editing in 8HUM zygotes (Taconic Biosciences GmbH, Germany). Breeding of mice from this process was carried out to re-generate 8HUM mice with a homozygous deletion of Rag2-8HUM_Rag2^{-/-}—rendering the line immunodeficient for xenograft studies.

### 8HUM_Rag2^{-/-}

### Rag-Recombination Activating Gene 2

located on mouse chromosome 2
Ensembl gene ID: ENSMUSG00000032864
NCBI gene ID: 19374

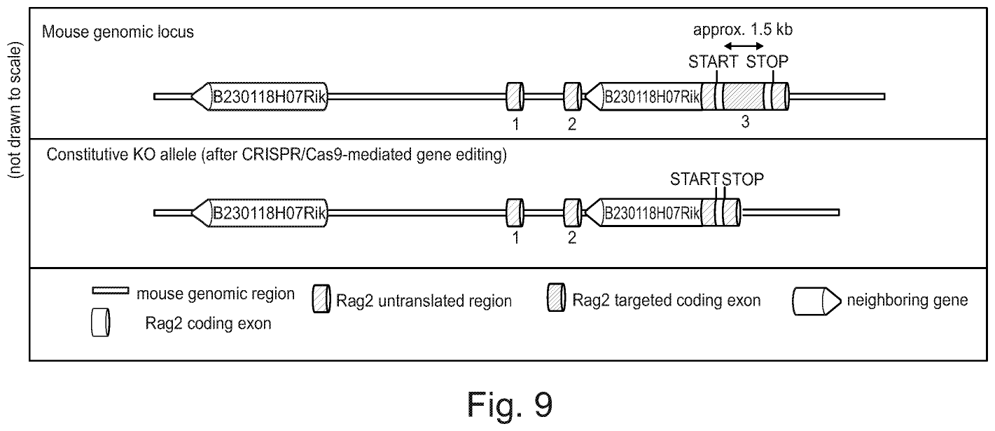

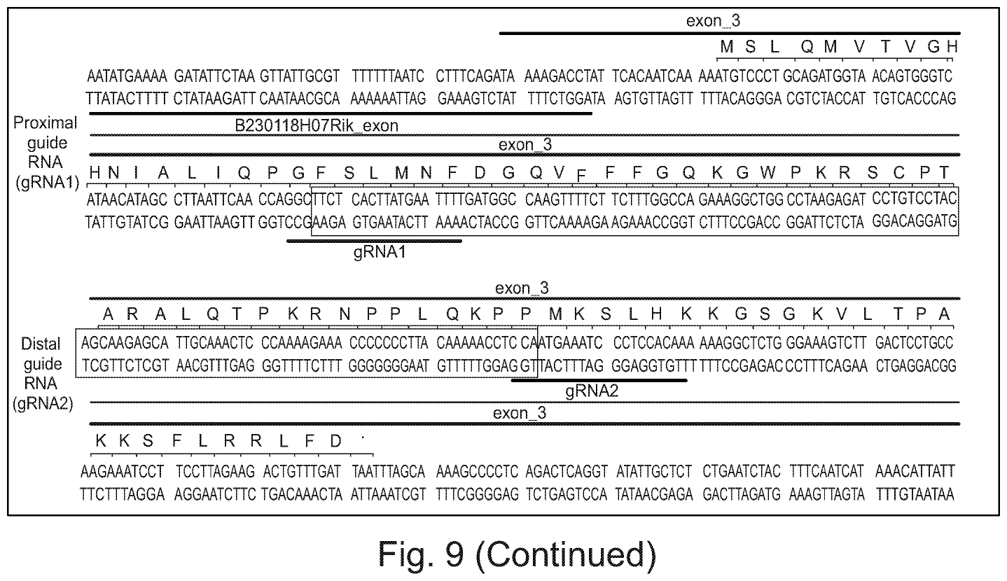

The targeting strategy is based on NCBI transcript NM_009020.3 which corresponds to Ensembl transcript ENSMUST00000044031. The Cas9 protein along with the proximal and distal gRNAs are injected into 8HUM zygotes and the constitutive knock-out allele is obtained after CRISPR/Cas9-mediated gene editing (see FIG. 9).

Mice received from Taconic were inter-crossed until the desired genotype was achieved.

### Final Designation: 8HUM_Rag2^{-/-}

C57BL/6-Nr1i2^{tm1198(NR112)Arte}-Nr1i3^{tm1089(NR113)Arte}-Cyp3a^{tm1109(CYP3A4/3A7)Arte}-Cyp3a13^{tm1728Arte}-Cyp2d^{tm1836(CYP2D6*2)Arte}-Cyp2c^{tm1897(albCYP2C9)Arte}-CYP1a1^{tm2025(CYP1A1)Arte}-Cyp1a2^{tm2024(CYP1A2)Arte} Rag2^{em6431Tac}

As with the 8HUM line, the 8HUM_Rag2^{-/-} line was maintained as ‘heterozygous’ at the CYP2C9/Cyp2c locus—i.e. one allele is the mouse Cyp2c cluster, the other is deleted for Cyp2c and humanised for CYP2C9.

Animals were on a C57BL/NTac background and were bred, and experimental work carried out in, the Medical School Resource Unit, University of Dundee. Mice were held at positive pressure in Techniplast Sealsave BlueLine micro-isolator cages, with Eco-Pure chip7D bedding (Datesand Group, UK) and ad libitum access to water and food—RM1 for maintenance, RM3 for breeding (Special Diet Services, UK). Temperature (20° C.-24° C.) and relative humidity (45%-65%) were maintained in a 12-hour light-dark environment.

All animal work was approved by the Welfare and Ethical treatment of Animals Committee, under Home Office Project (PAFCCC160) and Personal licences (I94242D3D, IDFA32717, I372C0F97) under the Animals (Scientific Procedures) Act 1986, as amended by EU Directive 2010/63/EU. Animals were inspected regularly by trained and experienced staff, with 24-hour access to veterinary advice.

On study completion, animals were sacrificed by exposure to a rising concentration of CO₂ and death confirmed by exsanguination, according to Schedule 1 of the Animals (Scientific Procedures) Act 1986.

### Experimental Design

Adult female (>8<22 weeks) mice were randomly allocated into control or experimental groups and allowed to adapt to their social setting for 7d before study start. Cages were adjacent to each other on the same level of a ventilated rack, in the same room, for the study duration.

Neither animal staff nor experimenters were blinded to the identity of the mice or the experimental group in which they were placed, before, during or after the study.

Sample size: group sizes of n=3-5 were used, following consideration of power calculations using G*Power (18), with an effect size of 1.75 and power of 80%.

Data analysis: Dependencies of calculated tumour volumes versus time were analysed by non-linear regression using exponential growth and exponential decay functions (Prism 5 software, Graphpad, US). Rate constants in both functions were constrained to positive values to maintain consistency with the function name. Values for plateau parameters in exponential decay function were set to zero.

### Protocol

A375 human melanoma cells (ATCC: CRL-1619; RRID: CVCL_UD29) and 5555 murine melanoma cells (19-21) were grown as directed, with the latter subject to commercial murine pathogen testing (IDEXX Bioanalytics GmbH, Germany) and both to in-house mycoplasma testing (MycoAlert Mycoplasma Detection kit, Lonza Rockland, USA) before use. Passage number was recorded for each study.

Cell lines were harvested on the morning of the study, kept on ice and transferred to the animal facility for use within 1h. All animal work was carried out in the sterile environment of a Tecniplast Changing station.

Mice were weighed, fur removed on one/both flanks by electric shaver and placed individually in a red plastic inhalation chamber connected to an anaesthetic machine (Vet-Tech Solutions, Congleton, UK), to which was connected an anaesthetic maintenance tube running into the changing station. General anaesthesia was induced using an isoflurane (Piramal Critical Care, UK)/oxygen mixture in a Series 3 vapouriser (O₂ flow rate 2l/min, isoflurane 3.5-4%) and maintained when the mouse was removed from the chamber by lying the animal on its front, snout placed just inside the end of the anaesthetic maintenance tube and the isoflurane/oxygen flow switched to the tube (O₂ flow rate 1.5l/min, isoflurane 1.5-2.5%). Prepared cells (3.5-5×10⁶, 100 μl in DMEM (Thermofisher Scientific, UK) were taken up in a 1 ml plastic syringe and injected subcutaneously (s.c.) to one or both flanks using a 25 mm/23G needle. [Optional: cells can be re-suspended in ECM (Sigma), diluted 1:1 with DMEM]. This procedure routinely took <3 min. Immediately after injection, the mouse was returned to its home cage, placed on its front and monitored during recovery, which routinely took <5 min. [Optional: s.c. injection may be carried out immediately after removing mouse from the inhalation chamber, while still under general anaesthesia. However, particularly with immortalised human tumours, care must be taken to avoid self-injection; on safety grounds the maintenance anaesthesia route is strongly recommended.]

In addition to routine welfare monitoring, mice were weighed and checked for tumour growth daily. Body weight was used in conjunction with a body scoring system (22). Deviation from normal health, >10% body weight loss, or a body condition score of 2 or less was referred to the NVS or NACWO. If any animal appeared distressed, or a tumour ulcerated, the animal was removed from study and killed by a Schedule 1 method.

Once established, tumours were measured twice in two dimensions (maximum breadth and length) using digital callipers, by the same person to avoid interindividual variation. Treatment was also started at this point, mice receiving either vehicle or drug daily (p.o.).

Dabrafenib methanesulfonate (LC Laboratories, MA, USA) was prepared as a 6.3 mg/ml suspension in vehicle (0.5% (w/v) hydroxypropylmethylcellulose, 0.2% (v/v) Tween-80) after 10 min sonication in a water bath and administered daily (p.o.) at 5 ml/kg, and a dose of 31.5 mg/kg. This is equivalent to approximately 150 mg of dabrafenib base for a 70 kg human (23), approximately half of the recommended daily dose (24).

Trametinib (LC Laboratories, MA, USA) was prepared as a 0.07048 mg/ml suspension in vehicle (0.5% (w/v) hydroxypropylmethylcellulose, 0.5% (v/v) Tween-20) and administered daily (p.o.) at 5 ml/kg, and a dose of 0.3524 mg/kg. This is equivalent to 2 mg of trametinib for a 70 kg human (23), which is the recommended daily dose (25).

Tumour volume was estimated using the formula ((width*width)*length)/2 (26). Tumour length was also monitored, and mice in which tumour length reached 15 mm (either individually or in total if tumours on both flanks) were sacrificed by a Schedule 1 method and blood, tissues and tumours harvested as appropriate for downstream analysis.

### Example 1: Human Xenografts can Successfully be Grown in Rag2^{null}/8HUM Murine Models

### Introduction

To investigate the suitability of the immunodeficient 8HUM model for xenograft cancer studies, the ability of Rag2^{null}/8HUM and 8HUM models to grow murine and human cell lines was investigated. The suitability of the 8HUM to grow syngeneic tumours was also confirmed.

### Materials and Methods

Rag2^{null}/8HUM and 8HUM (N=3 of each cohort) were injected with BRAFV600E human melanoma A375 cells. 8HUM mice were also injected with GM5555 murine melanoma cell and treated daily with vehicle or Dabrafenib every day for 15 days and tumour growth measured. 8HUM mice were used to determine syngeneic growth of the murine melanoma cell line, 5555, derived from a C57BL/6_BRAF+/LSL-BRAFV600E; Tyr::CreERT2+/o transgenic model (19) and reported by Hirata et al. as being sensitive to the selective BRAF inhibitor, and vemurafenib precursor, PLX4720 (27) in vitro, but refractory to this drug in vivo (26). More recently, the second-generation mutant BRAF inhibitor dabrafenib (in combination with the MEK inhibitor trametinib) has become standard of care in UK and Europe in the treatment of unresectable or metastatic BRAFV600 mutant melanoma (28, 29).

### Results

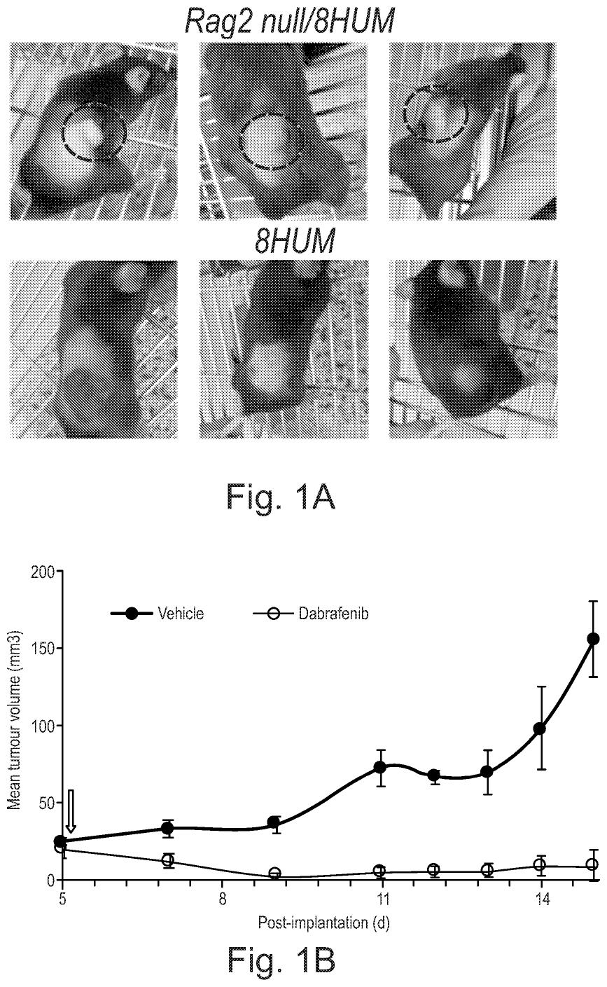

As shown in FIG. 1, 5555 cells were injected s.c. into one flank of adult female 8HUM mice, divided into two groups of five mice. After 5 days tumours had established in each animal and were measurable, at which point daily oral treatment with either vehicle or dabrafenib was started. While tumours in vehicle-treated mice continued to grow over the following 2 weeks, over the same period tumours in mice treated with dabrafenib became almost undetectable.

Tumour growths were observed in Rag2^{null}/8HUM mice injected with BRAFV600E human melanoma A375 cells (FIG. 1A). The immunocompetent 8HUM mice were not capable of supporting growth of A375 cells and no growths were observed (FIG. 1A). The immunocompetent 8HUM mice were able to support syngeneic tumour growth of GM5555 murine melanoma cells. The growth of GM5555 could be successfully inhibited by daily treatment with Dabrafenib (FIG. 1B).

These data demonstrate that C57BL/6-origin tumours can be grown in a syngeneic manner, and that the BRAFV600 mutant murine melanoma cell line is exquisitely sensitive to the BRAF inhibitor dabrafenib. These results confirm that the immunodeficient Rag2^{null}/8HUM mouse model is a suitable background for human xenografts, and therefore pharmacodynamic and mechanistic drug studies can be performed on human derived cells in this model.

### Example 2: The Rag2^{null}/8HUM Murine Model is a Suitable Background for Drug Efficacy Studies

### Introduction

To establish whether the Rag2^{null}/8HUM murine model is a suitable background for studying drug efficacy on human tumour xenografts, Rag2^{null}/8HUM mice with BRAFV600E human melanoma A375 tumours were subjected to treatment with Trametinib or Dabrafenib and the effect on tumour volume evaluated.

### Materials and Methods

Adult female Rag2^{null}/8HUM mice (n=3) were transplanted with BRAFV600E human melanoma A375 cells. Mice were treated daily with vehicle or Trametinib from day 20, and stopped on day 30 (arrow). The mean tumour volume (mm³) was measured throughout the experiment.

The same experiment was performed with adult female Rag2^{null}/8HUM mice (N=3) treated with vehicle or Dabrafenib from day 28 following transplant with BRAFV600E human melanoma A375 cells and stopped on day 35. The mean tumour volume (mm³) was measured throughout the experiment.

### Results

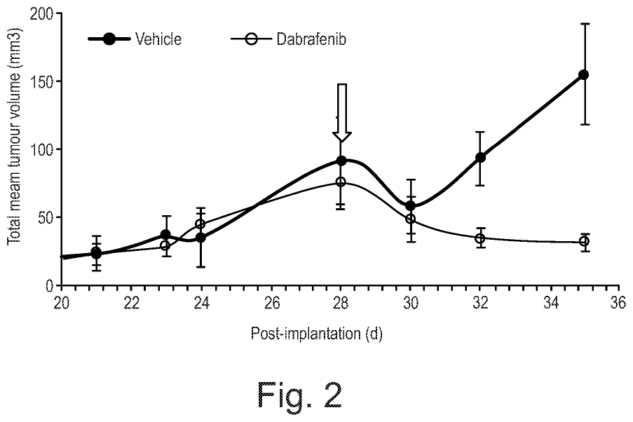

By creating an immunodeficient variant of the 8HUM mouse line, where the Rag2 locus is deleted, we extended work to xenografts with the BRAF mutant human melanoma cell line, A375. FIG. 2 shows change in total mean tumour volume following s.c. injection of A375 cells injected into both flanks of adult female 8HUM_Rag2^{-/-} mice. Daily treatment of these mice started 28d after injection of cells, either with vehicle or dabrafenib (arrow); while the tumours in the former group continued to grow, tumours in the mice treated with the BRAF inhibitor shrank in size until by d35 (at which point the vehicle-treated mice had to be sacrificed due to tumour size) there was a significant difference in the treatment effect between the two groups (FIG. 2). The data from vehicle group follows the exponential growth dependency and does not fit with the exponential decay function. Conversely, the dabrafenib group data follows exponential decay dependency and does not fit the exponential growth function. These data clearly demonstrate not only that it is possible to grow a human melanoma cell line as a xenograft in this immunodeficient version of the 8HUM mouse model, but it is also possible to demonstrate sensitivity of A375 tumours to BRAF inhibitors.

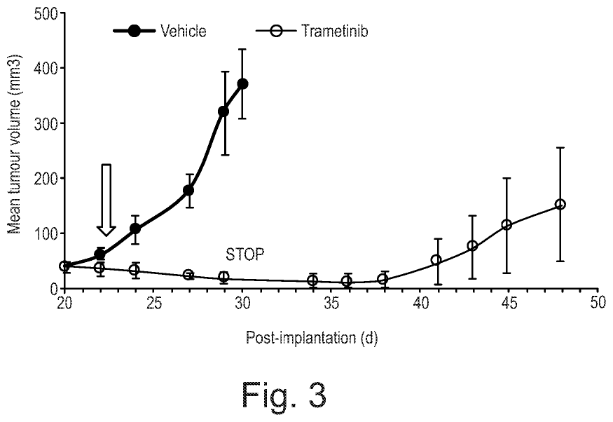

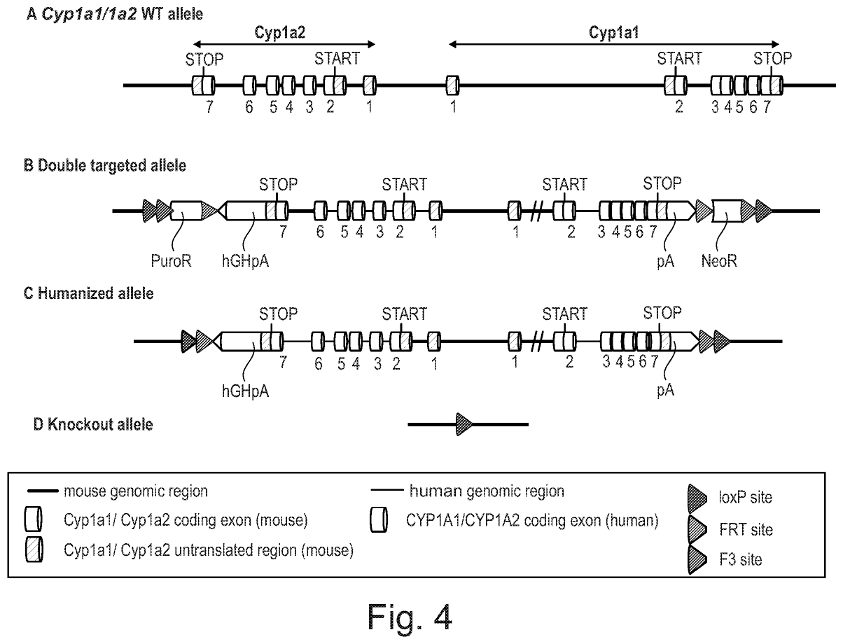

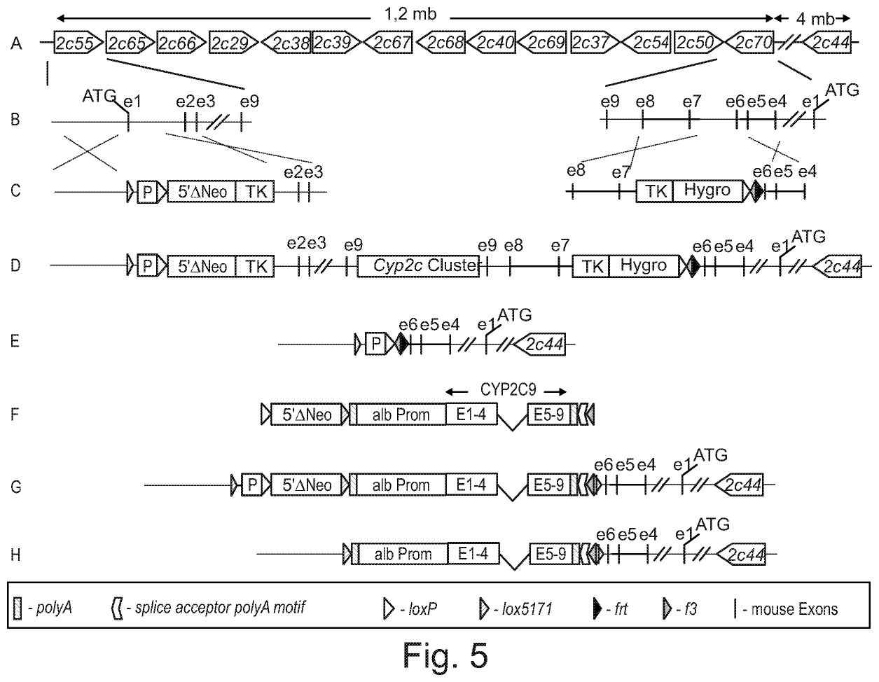

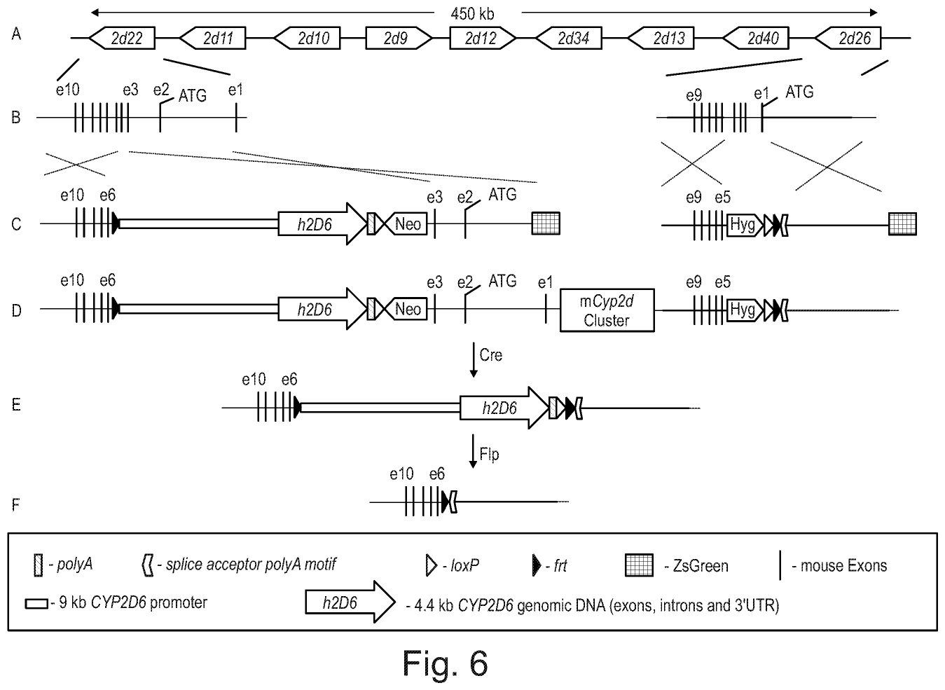

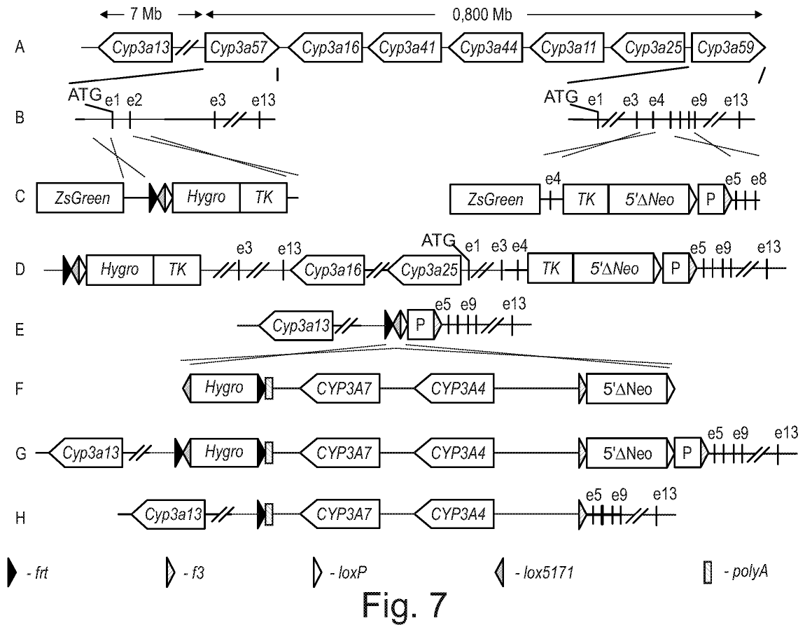

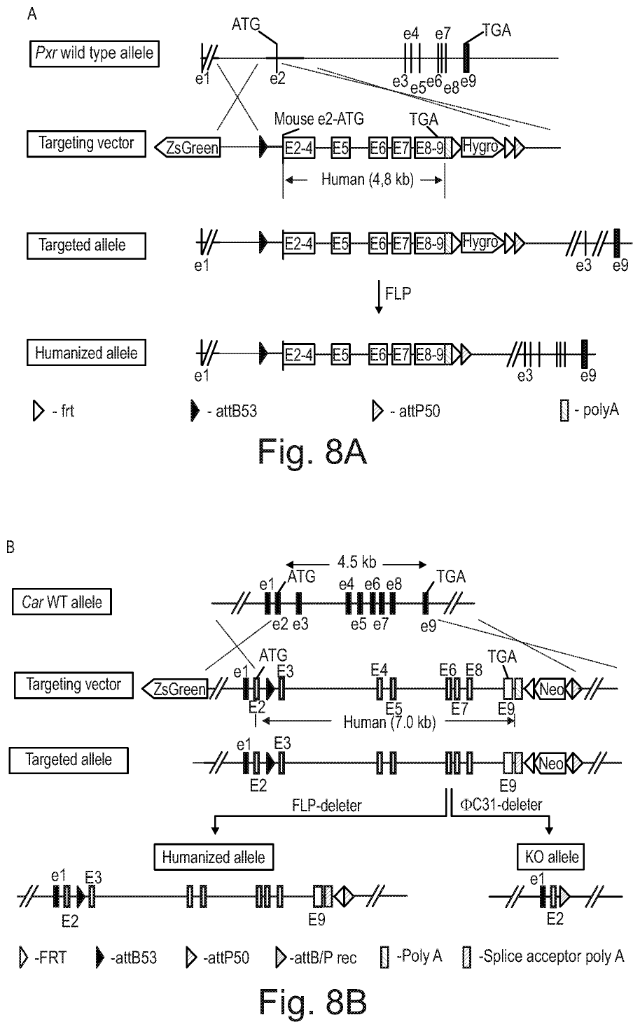

Dabrafenib is used in a clinical setting in combination with the MEK inhibitor trametinib. We tested the ability of trametinib to stop tumour growth, using A375 cells injected s.c. in the flank of adult female 8HUM_Rag2^{-/-} mice (FIG. 3). Daily treatment with vehicle or drug commenced on d22 after cell injection (arrow), and in the following period tumours in mice treated with vehicle continued to grow until by d30 they had reached the maximum size permitted. At this point (FIG. 3, STOP) tumours in the 8HUM_Rag2^{-/-} mice had regressed to the point where they were essentially undetectable, and trametinib treatment was stopped. Interestingly, over the following 10 days, tumours began to regrow in the absence of treatment until they were again palpable and measurable, and continued to grow over the next week or so until the study was terminated on d48 (FIG. 3).

### CONCLUSIONS/DISCUSSION

8HUM mouse and its immunodeficient variant 8HUM_Rag2^{-/-} are capable of hosting both syngeneic tumours and xenografts, respectively.

While numbers of animals used in syngeneic or xenograft work in pre-clinical drug development are difficult to assess, the total will be significant given the number of drug candidates being tested across Pharma at any given time, and such growth of tumours in vivo is also carried out in other research areas, for example toxicology. A PubMed search for papers published in 2019 found ˜5,000 papers containing ‘xenograft’ in the title or abstract, illustrating the extent to which the 8HUM and 8HUM_Rag2^{-/-} models—modified as appropriate by gene editing to recapitulate disease models—may be able to address 3Rs issues by both refinement: generation of better, more human-relevant data, and reduction—use of fewer animals without loss of statistical power. Pre-clinical use of humanised models to prevent failure of a drug candidate during clinical testing because of species differences in drug disposition would undoubtedly save significant numbers of mice. They will also allow complex drug combinations to be tested and treatment regimens optimised in a manner which is not feasible by clinical trial, and reduce the chances of drug-drug interactions.

### Example 3-Rag2/II2Rγ 8HUM Mouse Model

### Introduction

It is well known that the more genetically complex model is, the lower the viability of the offspring. Indeed, the 8HUM mouse as developed in Henderson et al., are maintained by crossing males homozygous for the gene deletions and humanization with females heterozygous for the Cyp2c gene cluster deletion/CYP2C9 humanization and retention of one allele of the murine Cyp2c gene cluster due to high mortality of homozygous female mice. Therefore, it was surprising to the inventors that the Rag2 8Hum mouse was indeed viable with a further mutation introduced. The inventors also sought to produce a Rag2/IL2Rγ double CRISPR knockout 8HUM mouse.

### Method

IL2Rg-interleukin 2 receptor, gamma chain.
   located on mouse chromosome X
   Ensembl gene ID: ENSMUSG00000031304
   NCBI gene ID: 16186

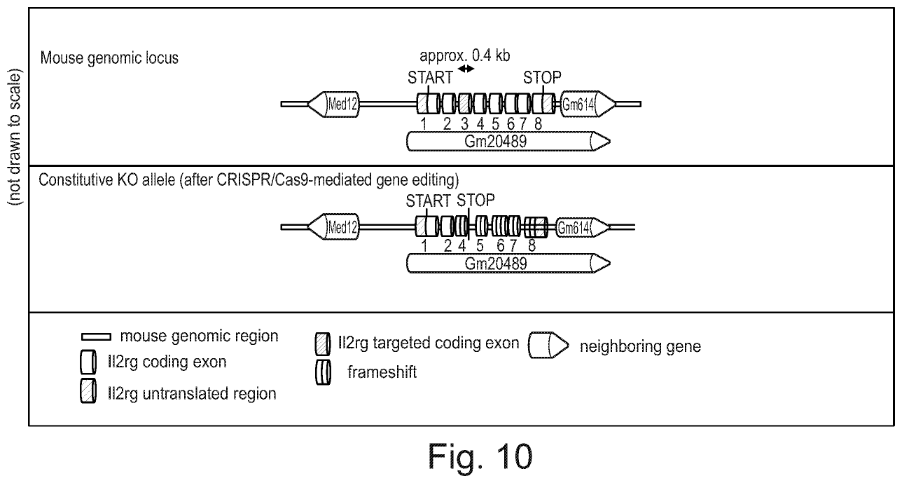

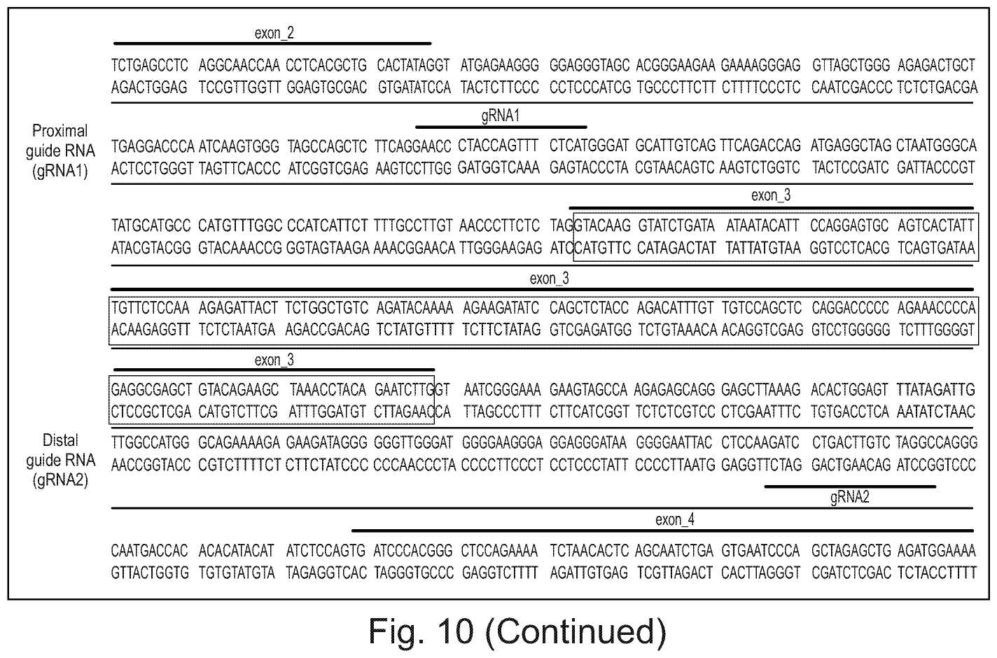

The targeting strategy is based on NCBI transcript NM_013563.4 which corresponds to Ensembl transcript ENSMUST00000033664.13. The Cas9 protein along with the proximal and distal gRNAs were injected into DND0119 zygotes and the constitutive Knock-Out allele is obtained after CRISPR/Cas9-mediated gene editing (see FIG. 10).

Mice received from Taconic were inter-crossed until the desired genotype was achieved.
Final designation: 8HUM_IL2Rg2^{-/-}
C57BL/6-Nr1i2^{tm1198(NR112)Arte}-Nr1i3^{tm1089(NR113)Arte}-Cyp3a^{tm1109(CYP3A4/3A7)Arte}-Cyp3a13^{tm1728Arte}-Cyp2d^{tm1836(CYP2D6*2)Arte}-Cyp2c^{tm1897(albCYP2C9)Arte}-CYP1a1^{tm2025 (CYP1A1)Arte}-Cyp1a2^{tm2024(CYP1A2)Arte} Il2rg^{em6432Tac}

As with the 8HUM line, the 8HUM_IL2Rg^{-/-} line was maintained as ‘heterozygous’ at the CYP2C9/Cyp2c locus—i.e. one allele is the mouse Cyp2c cluster, the other is deleted for Cyp2c and humanised for CYP2C9.

Double immunodeficient 8HUM line: 8HUM_Rag2^{-/-}_IL2Rg^{-/-} Mice received from Taconic were inter-crossed until the desired genotype was achieved.
C57BL/6-Nr1i2^{tm1198(NR112)Arte}-Nr1i3^{tm1089(NR113)Arte}-Cyp3a^{tm1109(CYP3A4/3A7)Arte}-Cyp3a13^{tm1728Arte}-Cyp2d^{tm1836(CYP2D6*2)Arte}-Cyp2c^{tm1897(albCYP2C9)Arte}-CYP1a1^{tm2025 (CYP1A1)Arte}-Cyp1a2^{tm2024(CYP1A2)Arte} Rag2^{em6431Tac} Il2rg^{em6432Tac}

### Results

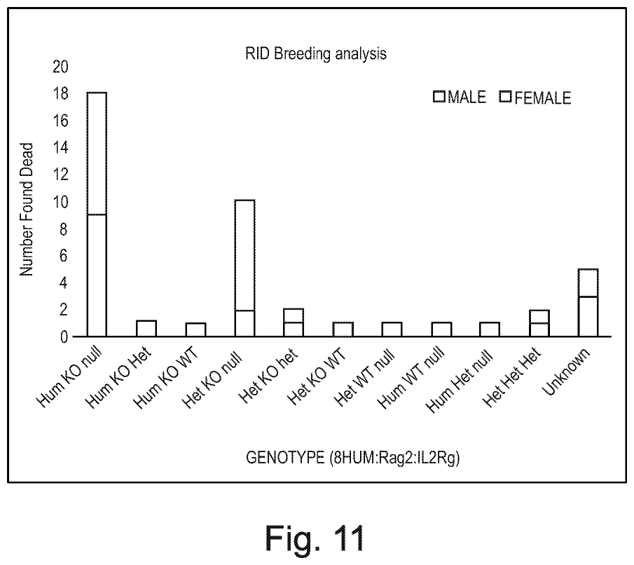

As summarised below (Tables 1 and 2) and in FIG. 11, in the more complex immune deprived line there was high level of mortality.

### Summary of Mouse Survival:

TABLE 1
Overview of Breeding Pairs and Deaths

| Breeding | 8HUM: RAG2: | | Found dead/total |
|---|---|---|---|
| Pair | IL2Rg | 8HUM: RAG2: IL2Rg | born |
| PAIR | MALE | FEMALE | DEATHS |
| A | HUM: KO: y/+ | HET: WT: +/− | 2/13 |
| D | HUM: KO: y/− | HET: KO: +/+ | 1/17 |
| E | HET: WT: y/− | HET: HET: +/+ | 0/19 |
| F | HUM: KO: y/+ | HET: WT: +/− | 0/19 |
| G | HUM: KO: y/+ | HET: WT: +/− | 0/5 |
| H | HUM: KO: y/+ | HET: HET: +/− | 2/20 |
| J | HUM: HET: y/− | HET: HET: +/− | 1/17 |
| K | HUM: HET: y/− | HET: HET: +/− | 1/9 |
| L | HUM: HET: y/− | HET: HET: +/− | 0/23 |
| N | HUM: KO: y/− | HET: KO: −/− | 6/23 |
| P | HUM: HET: y/− | HET: HET: +/− | 1/11 |
| Q | HUM: HET: y/− | HET: HET: −/− | 0/6 |
| R | HUM: HET: y/− | HET: HET: −/− | 2/24 |
| S | HUM: HET: y/− | HET: HET: −/− | 1/14 |
| T | HUM: KO: y/− | HET: KO: −/− | 13/26 |
| V | HUM: HET: y/− | HET: KO: −/− | No pups |
| W | HUM: HET: y/− | HET: KO: −/− | 3/7 |
| X | HUM: KO: y/− | HET: KO: +/− | 2/14 |
| Y | HUM: KO: y/− | HET: KO: +/− | 1/6 |
| Z | HUM: KO: y/− | HET: HET: +/− | 2/13 |
| AA | HUM: KO: y/− | HET: KO: −/− | 8/8 |
| AB | HUM: KO: y/− | HET: KO: −/− | No pups |
| AC | HUM: KO: y/− | HET: KO: −/− | 1/6 |
| AD | HUM: KO: y/− | HET: KO: −/− | No pups |
| AE | HUM: KO: y/− | HET: KO: −/− | No pups |
| AF | HUM: KO: y/− | HET: KO: −/− | No pups |

TABLE 2
Summary of Deaths According to Genotype

| Genotype | Found Dead | | |
|---|---|---|---|
| 8HUM: RAG2: | | | Total |
| IL2Rg | Male | Female | deaths |
| Hum KO null | 9 | 9 | 18 |
| Hum KO Het | | 1 | 1 |
| Hum KO WT | 1 | | 1 |
| Het KO null | 2 | 8 | 10 |
| Het KO het | 1 | 1 | 2 |
| Het KO WT | | 1 | 1 |
| Het WT null | 1 | | 1 |
| Hum WT null | 1 | | 1 |
| Hum Het null | | 1 | 1 |
| Het Het Het | 1 | 1 | 2 |
| Unknown | 3 | 2 | 5 |

### Discussion

High levels of mortality were observed with the Rag2/IL2Rγ double immunodeficient 8HUM line. Despite high mortality with the double immunodeficient 8HUM line, the inventors were surprised to find that an 8Hum mouse can be made immunodeficient by genetic deletion of a single gene such as Rag2 and mortality remains low.

Sequences
Rag2^{-/-} mouse
gRNA1:
(SEQ ID NO: 1)
5'-GGCTTCTCACTTATGAATTT-3'
(SEQ ID NO: 2)
3'-CCGAAGAGTGAATACTTAAA-5'
gRNA2:
(SEQ ID NO: 3)
5'-CCAATGAAATCCCTCCACAA-3'
(SEQ ID NO: 4)
3'-GGTTACTTTAGGGAGGTGTT-5'
IL2Rg^{-/-} mouse
gRNA1:
(SEQ ID NO: 5)
5'-GAACCCTACCAGTTTCTCAT-3'
(SEQ ID NO: 6)
3'-CTTGGGATGGTCAAAGAGTA-5'
gRNA2:
(SEQ ID NO: 7)
5'-AGATCCTGACTTGTCTAGGC-3'
(SEQ ID NO: 8)
3'-TCTAGGACTGAACAGATCCG-5'

### REFERENCES

1. Hingorani, A. D., Kuan, V., Finan, C. et al. Improving the odds of drug development success through human genomics: modelling study. Sci Rep 9, 18911 (2019). https://doi.org/10.1038/s41598-019-54849-w
2. Slaughter R L, Edwards DJ. Recent advances: the cytochrome P450 enzymes. Ann Pharmacother. 1995; 29:619-24.
3. Mackowiak B, Hodge J, Stern S, and Wang H (2018) The roles of xenobiotic receptors: beyond chemical disposition. Drug Metab Dispos 46:1361-1371.
4. van Waterschoot R A, van Herwaarden A E, Lagas J S, Sparidans R W, Wagenaar E, van der Kruijssen C M, Goldstein J A, Zeldin D C, Beijnen J H, and Schinkel A H (2008) Midazolam metabolism in cytochrome P450 3A knockout mice can be attributed to up-regulated CYP2C enzymes. Mol Pharmacol 73:1029-1036.
5. Wallace H A, Marques-Kranc F, Richardson M, Luna-Crespo F, Sharpe J A, Hughes J, Wood W G, Higgs D R, Smith A J. Manipulating the mouse genome to engineer precise functional syntenic replacements with human sequence. Cell. 2007 Jan. 12; 128 (1): 197-209. doi: 10.1016/j.cell.2006.11.044. PMID: 17218265.
6. Mashimo, Tomoji et al. Generation and Characterization of Severe Combined Immunodeficiency Rats. Cell Reports, Volume 2, Issue 3, 685-694.
7. Honek J. Preclinical research in drug development. Medical Writing. 2017; 26:5-8.
8. Dowden H, Munro J. Trends in clinical success rates and therapeutic focus. Nat Rev Drug Discov. 2019; 18 (7): 495-6.
9. Harrison R K. Phase II and phase III failures: 2013-2015. Nat Rev Drug Discov. 2016; 15 (12): 817-8.
10. Long J E, Jankovic M, Maddalo D. Drug discovery oncology in a mouse: concepts, models and limitations. Future Sci OA. 2021; 7 (8): FSO737.
11. Sun D, Gao W, Hu H, Zhou S. Why 90% of clinical drug development fails and how to improve it? Acta Pharmaceutica Sinica B. 2022.
12. Henderson C J, Kapelyukh Y, Scheer N, Rode A, McLaren A W, Macleod A K, et al. An Extensively Humanized Mouse Model to Predict Pathways of Drug Disposition and Drug/Drug Interactions, and to Facilitate Design of Clinical Trials. Drug Metab Dispos. 2019; 47(6): 601-15.
13. Duarte D, Vale N. Evaluation of synergism in drug combinations and reference models for future orientations in oncology. Curr Res Pharmacol Drug Discov. 2022; 3:100110.
14. Kersten K, de Visser K E, van Miltenburg M H, Jonkers J. Genetically engineered mouse models in oncology research and cancer medicine. EMBO Mol Med. 2017; 9(2): 137-53.
15. Byrne A T, Alferez D G, Amant F, Annibali D, Arribas J, Biankin A V, et al. Interrogating open issues in cancer precision medicine with patient-derived xenografts. Nat Rev Cancer. 2017; 17(4): 254-68.
16. Goto T. Patient-Derived Tumor Xenograft Models: Toward the Establishment of Precision Cancer Medicine. J Pers Med. 2020; 10(3).
17. Dudova Z, Conte N, Mason J, Stuchlik D, Pesa R, Halmagyi C, et al. The EurOPDX Data Portal: an open platform for patient-derived cancer xenograft data sharing and visualization. BMC Genomics. 2022; 23(1): 156.
18. Faul F, Erdfelder E, Buchner A, Lang A G. Statistical power analyses using G*Power 3.1: tests for correlation and regression analyses. Behav Res Methods. 2009; 41(4): 1149-60.
19. Dhomen N, Reis-Filho J S, da Rocha Dias S, Hayward R, Savage K, Delmas V, et al. Oncogenic Braf induces melanocyte senescence and melanoma in mice. Cancer Cell. 2009; 15(4): 294-303.
20. Hirata E, Girotti M R, Viros A, Hooper S, Spencer-Dene B, Matsuda M, et al. Intravital imaging reveals how BRAF inhibition generates drug-tolerant microenvironments with high integrin beta1/FAK signaling. Cancer Cell. 2015; 27(4): 574-88.
21. Roulstone V, Pedersen M, Kyula J, Mansfield D, Khan A A, McEntee G, et al. BRAF- and MEK-Targeted Small Molecule Inhibitors Exert Enhanced Antimelanoma Effects in Combination With Oncolytic Reovirus Through ER Stress. Mol Ther. 2015; 23(5): 931-42.
22. Ullman-Cullere M H, Foltz C J. Body condition scoring: a rapid and accurate method for assessing health status in mice. Lab Anim Sci. 1999; 49(3): 319-23.
23. Reagan-Shaw S, Nihal M, Ahmad N. Dose translation from animal to human studies revisited. FASEB J. 2008; 22(3): 659-61.
24. Medicine NLo. NIH; [LABEL: TAFINLAR-dabrafenib capsule; The DailyMed database contains labeling for drugs and biological products submitted to the Food and Drug Administration (FDA)]. Available from: https://dailymed.nlm.nih.gov/dailymed/drugInfo.cfm?setid=fee1e6b1-e1a5-4254-9f2e-a70e0f8dbdea.
25. Medicine NLo. DailyMED: NIH; [LABEL: MEKINIST-trametinib tablet, film coated; The DailyMed database contains labeling for drugs and biological products submitted to the Food and Drug Administration (FDA)]. Available from: https://dailymed.nlm.nih.gov/dailymed/drugInfo.cfm?setid=0002ad27-779d-42ab-83b5-bc65453412a1.
26. Faustino-Rocha A, Oliveira P A, Pinho-Oliveira J, Teixeira-Guedes C, Soares-Maia R, da Costa R G, et al. Estimation of rat mammary tumor volume using caliper and ultrasonography measurements. Lab Anim (NY). 2013; 42(6):217-24.
27. Tsai J, Lee J T, Wang W, Zhang J, Cho H, Mamo S, et al. Discovery of a selective inhibitor of oncogenic B-Raf kinase with potent antimelanoma activity. Proc Natl Acad Sci USA. 2008; 105(8): 3041-6.
28. Long G V, Stroyakovskiy D, Gogas H, Levchenko E, de Braud F, Larkin J, et al. Dabrafenib and trametinib versus dabrafenib and placebo for Val600 BRAF-mutant melanoma: a multicentre, double-blind, phase 3 randomised controlled trial. The Lancet. 2015; 386(9992):444-51.
29. NICE. Melanoma: assessment and management 2015 [updated 29 Jul. 2015. NICE guideline [NG14]. Available from: https://www.nice.org.uk/guidance/ng14/chapter/1-Recommendations #managing-stage-iii-melanoma-2.

1. An immunodeficient non-human animal wherein at least one endogenous drug metabolism enzyme has been substantially inactivated.

2. The immunodeficient non-human animal according to claim 1, wherein the at least one endogenous drug metabolism enzyme is substantially inactivated by substantially inactivating at least one gene encoding a drug metabolism enzyme.

3. The immunodeficient non-human animal according to claim 2, wherein the at least one endogenous gene encodes an enzyme of the endogenous cytochrome P450 monooxygenase system.

4. The immunodeficient non-human animal according to claim 2, wherein the at least one endogenous gene encodes any one or more genes comprised in the Cyp1a, Cyp2c, Cyp2d, or Cyp3a gene subfamilies.

5. The immunodeficient non-human animal according to claim 4, wherein the at least one endogenous gene encodes any one or more of Cyp1a1, Cyp1a2, Cyp2c55, Cyp2c65, Cyp2c66, Cyp2c29, Cyp2c38, Cyp2c39, Cyp2c67, Cyp2c68, Cyp2c40, Cyp2c69, Cyp2c37, Cyp2c44, Cyp2c54, Cyp2c50, Cyp2c70, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2d1, Cyp2d2, Cyp2d3, Cyp2d4, Cyp2d5 Cyp2d22, Cyp2d11, Cyp2d10, Cyp2d9, Cyp2d12, Cyp2d34, Cyp2d13, Cyp2d40, Cyp2d26, Cyp3a1, Cyp3a2, Cyp3a9, Cyp3a18, Cyp3a23, Cyp3a62, Cyp3a73, Cyp3a13, Cyp3a11, Cyp3a16, Cyp3a25, Cyp3a41, Cyp3a44, Cyp3a57, Cyp3a58-ps, Cyp3a59 or functionally equivalent orthologues and homologues.

6. The immunodeficient non-human animal according to claim 1, wherein genes encoding endogenous transcription factors Car or Pxr are substantially inactivated, optionally wherein both Car and Pxr are substantially inactivated.

7. The immunodeficient non-human animal according to claim 6, wherein the Cyp1a, Cyp2c, Cyp2d, and Cyp3a gene subfamilies and Pxr and Car are substantially inactivated.

8. The immunodeficient non-human animal according to claim 6, wherein Cyp1a1, Cyp1a2, Cyp2c29, Cyp2c37, Cyp2c38, Cyp2c39, Cyp2c40, Cyp2c44, Cyp2c50, Cyp2c54, Cyp2c55, Cyp2c65, Cyp2c66, Cyp2c67, Cyp2c68, Cyp2c69, Cyp2c70, Cyp2d9, Cyp2d10, Cyp2d11, Cyp2d12, Cyp2d13, Cyp2d22, Cyp2d26, Cyp2d34, Cyp2d40, Cyp3a11, Cyp3a13, Cyp3a16, Cyp3a25, Cyp3a41, Cyp3a44, Cyp3a57, Cyp3a59 and Pxr and Car are substantially inactivated.

9. The immunodeficient non-human animal according to claim 6, wherein Cyp1a1, Cyp1a2, Cyp2a2, Cyp2a3, Cyp2b2, Cyp2b3, Cyp2b12, Cyp2b15, Cyp2b21, Cyp2b31, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2c81, Cyp2c68, Cyp2c69, Cyp2c70, Cyp2d1, Cyp2d2, Cyp2d3, Cyp2d4, Cyp2d5, Cyp3a1, Cyp3a2, Cyp3a9, Cyp3a18, Cyp3a23, Cyp3a62, Cyp3a73, Cyp4a1, Cyp4a2, Cyp4a3, Cyp4a8 and Pxr and Car are substantially inactivated.

10. The immunodeficient non-human animal according to claim 1, wherein any one or more human genes selected from the list comprising: CYP1A1, CYP1A2, CYP1B1, CYP2A6, CYP2A13, CYP2A7, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, CYP3A43, CYP4A11, CYP4A22, CYP4B1, CYP4F2, CYP4F3, CYP4F8, CYP4F11, CYP4F12, CYP4F22, CYP4V2, CYP4X1, or CYP4Z1, or any functional equivalents thereof are expressed in the immunodeficient non-human animal

11. The immunodeficient non-human animal according to claim 10, wherein human CYP1A1, CYP1A2, CYP2C9, CYP2D6, CYP3A4 and CYP3A7, or any functional equivalents thereof are expressed in the immunodeficient non-human animal.

12. The immunodeficient non-human animal according to claim 1, wherein human CAR and/or PXR are expressed in the immunodeficient non-human animal.

13. The immunodeficient non-human animal according to claim 1, wherein the endogenous Cyp1a, Cyp2c, Cyp2d, and Cyp3a gene subfamilies and Pxr and Car are substantially inactivated, and human CYP1A1, CYP1A2, CYP2C9, CYP2D6, CYP3A4, CYP3A7, CAR and PXR are expressed the immunodeficient non-human animal.

14. The immunodeficient non-human animal according to claim 1, wherein the endogenous recombination activating gene 2 (Rag2) is substantially inactivated.

15. The immunodeficient non-human animal according to claim 1, wherein the immunodeficient non-human animal lacks functional T, B and/or NK cells.

16. The immunodeficient non-human animal according to claim 1, wherein the immunodeficient non-human animal is heterozygous for the endogenous Cyp2c subfamily gene cluster expression, wherein one allele of the endogenous Cyp2c subfamily gene cluster is substantially active.

17. The immunodeficient non-human animal according to claim 16, wherein the animal is female.

18. The immunodeficient non-human animal according to claim 1, wherein the immunodeficient non-human animal is an embryo, a neonate or an adult.

19. A cell isolated from an immunodeficient non-human animal according to claim 1.

20. The cell according to claim 19, wherein the cell is a germ cell or a somatic cell.

21. A method of performing ex vivo or in vitro drug studies, wherein the method comprises:
(i) providing a cell according to claim 19; optionally wherein the cell is maintained in cell culture;
(ii) administering to the cell at least one test compound; and
(iii) analysing at least one cellular characteristic.

22. A method of generating an immunodeficient non-human animal according to claim 1, wherein the method comprises substantially inactivating at least one endogenous drug metabolism enzyme in the immunodeficient non-human animal.

23. The method according to claim 22, wherein the method comprises substantially inactivating at least one or more endogenous drug metabolism enzymes from the list comprising: Cyp1a1, Cyp1a2, Cyp2c55, Cyp2c65, Cyp2c66, Cyp2c29, Cyp2c38, Cyp2c39, Cyp2c67, Cyp2c68, Cyp2c40, Cyp2c69, Cyp2c37, Cyp2c44, Cyp2c54, Cyp2c50, Cyp2c70, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2d1, Cyp2d2, Cyp2d3, Cyp2d4, Cyp2d5 Cyp2d22, Cyp2d11, Cyp2d10, Cyp2d9, Cyp2d12, Cyp2d34, Cyp2d13, Cyp2d40, Cyp2d26, Cyp3a1, Cyp3a2, Cyp3a9, Cyp3a18, Cyp3a23, Cyp3a62, Cyp3a73, Cyp3a13, Cyp3a11, Cyp3a16, Cyp3a25, Cyp3a41, Cyp3a44, Cyp3a57, Cyp3a58-ps, and Cyp3a59 or functionally equivalent orthologues and homologues.

24. The method according to claim 22, wherein the method comprises substantially inactivating endogenous transcription factors Car and/or Pxr, preferably both endogenous Car and Pxr are substantially inactivated.

25. The method according to claim 22, wherein the method comprises substantially inactivating Cyp1a1, Cyp1a2, Cyp2c29, Cyp2c37, Cyp2c38, Cyp2c39, Cyp2c40, Cyp2c44, Cyp2c50, Cyp2c54, Cyp2c55, Cyp2c65, Cyp2c66, Cyp2c67, Cyp2c68, Cyp2c69, Cyp2c70, Cyp2d9, Cyp2d10, Cyp2d11, Cyp2d12, Cyp2d13, Cyp2d22, Cyp2d26, Cyp2d34, Cyp2d40, Cyp3a11, Cyp3a13, Cyp3a16, Cyp3a25, Cyp3a41, Cyp3a44, Cyp3a57, Cyp3a59 and Pxr and Car in the immunodeficient non-human animal.

26. The method according to claim 22, wherein the method comprises substantially inactivating Cyp1a1, Cyp1a2, Cyp2a2, Cyp2a3, Cyp2b2, Cyp2b3, Cyp2b12, Cyp2b15, Cyp2b21, Cyp2b31, Cyp2c6, Cyp2c6, Cyp2c7, Cyp2c11, Cyp2c12, Cyp2c13, Cyp2c22, Cyp2c23, Cyp2c24, Cyp2c79, Cyp2c80, Cyp2c81, Cyp2c68, Cyp2c69, Cyp2c70, Cyp2d1, Cyp2d2, Cyp2d3, Cyp2d4, Cyp2d5, Cyp3a1, Cyp3a2, Cyp3a9, Cyp3a18, Cyp3a23, Cyp3a62, Cyp3a73, Cyp4a1, Cyp4a2, Cyp4a3, Cyp4a8 and Pxr and Car, in the immunodeficient non-human animal.

27. The method of claim 22, wherein the method further comprises introducing at least one DNA sequence encoding at least one human drug metabolising enzyme in the immunodeficient non-human animal.

28. The method according to claim 27, wherein the method further comprises introducing a plurality of DNA sequences encoding a plurality of human drug metabolising enzymes into the immunodeficient non-human animal.

29. The method according to claim 27, wherein the at least one DNA sequence encoding a human drug metabolism enzyme, encodes at least one or more of the following human drug metabolism enzymes: CYP1A1, CYP1A2, CYP1B1, CYP2A6, CYP2A13, CYP2A7, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, CYP3A43, CYP4A11, CYP4A22, CYP4B1, CYP4F2, CYP4F3, CYP4F8, CYP4F11, CYP4F12, CYP4F22, CYP4V2, CYP4X1, or CYP4Z1, or any functional equivalents thereof.

30. The method according to claim 27, wherein the method further comprises introducing at least one DNA sequence encoding human CAR or PXR, preferably both CAR and PXR.

31. The method according to claim 22, wherein the method comprises;
(i) substantially inactivating the endogenous gene clusters Cyp1a, Cyp2c, Cyp2d, and Cyp3a and endogenous Car and Pxr in the immunodeficient non-human animal;
(ii) introducing a plurality of DNA sequences encoding human CYP1A1, CYP1A2, CYP2C9, CYP2D6, CYP3A4, CYP3A7, PXR and CAR in the immunodeficient non-human animal.

32. A method of performing preclinical drug studies, wherein the method comprises:
(i) providing an immunodeficient non-human animal according to claim 1;
(ii) administering at least one test compound to the animal;
(iii) obtaining at least one biological sample from the animal, and/or recording clinical parameters of the animal; and where at least one biological sample has been obtained,
(iv) analysing the biological sample for at least one analyte.

33. A method of measuring rate of metabolism of a drug compound, wherein the method comprises:
(i) providing an immunodeficient non-human animal according to claim 1;
(ii) administering at least one test compound to the animal;
(iii) obtaining at least one biological sample from the animal, and/or recording clinical parameters of the animal; and where at least one biological sample has been obtained,
(iv) measuring the measuring the rate of metabolism of the compound in the biological sample.

34. The method according to claim 32, wherein the method comprises a further step of transplanting human cells into the immunodeficient non-human animal prior to performing step (ii).

35. The method according to claim 34, wherein the human cells are patient-derived cells, healthy donor cells or a cell line.

36. The method according to claim 35, wherein patient-derived cells are cancer cells.

37. A method of testing one or more pharmaceutical compounds, wherein the method comprises:
(i) providing an immunodeficient non-human animal according to claim 1;
(ii) administering at least one test compound to the animal; and
(iii) obtaining at least one biological sample from the animal, and/or recording clinical parameters of the animal; and optionally, where at least one biological sample has been obtained,
(iv) analysing the biological sample for at least one analyte or clinical parameter.

38. A method of testing anti-cancer compounds, wherein the method comprises:
(i) providing an immunodeficient non-human animal according to claim 1;
(ii) transplanting healthy and/or cancerous human cells into the animal;
(iii) obtaining at least one biological sample from the animal, and/or recording clinical parameters of the animal; and where at least one biological sample has been obtained,
(iv) analysing the biological sample for at least one analyte or clinical parameter.

39. A method of testing anti-malarial compounds, wherein the method comprises:
(i) providing an immunodeficient non-human animal according to claim 1;
(ii) infecting the animal with a Plasmodium parasite;
(iii) obtaining at least one biological sample from the animal, and/or optionally recording clinical parameters of the animal; and where at least one biological sample has been obtained,
(iv) analysing the biological sample for at least one analyte or clinical parameter.

40. The method according to claim 37, wherein the method comprises a further step of transplanting human cells into the immunodeficient non-human animal prior to, or simultaneously to, or immediately after, performing step (ii) of the method.

41. The method according to claim 39, wherein the Plasmodium parasite is any one of Plasmodium falciparum, Plasmodium vivax, Plasmodium ovale, Plasmodium malariae, or Plasmodium knowlesi.


SEQUENCE LISTING (8 sequences)

SEQ ID NO: 1 - RNA, 20 nt, synthetic construct
  source 1..20: mol_type = other RNA
  misc_feature 1..20: note = Rag2 gRNA1
ggcttctcacttatgaattt

SEQ ID NO: 2 - RNA, 20 nt, synthetic construct
  source 1..20: mol_type = other RNA
  misc_feature 1..20: note = Rag2 gRNA1 reverse complement
aaattcataagtgagaagcc

SEQ ID NO: 3 - RNA, 20 nt, synthetic construct
  source 1..20: mol_type = other RNA
  misc_feature 1..20: note = Rag2 gRNA2
ccaatgaaatccctccacaa

SEQ ID NO: 4 - RNA, 20 nt, synthetic construct
  source 1..20: mol_type = other RNA
  misc_feature 1..20: note = Rag2 gRNA2 reverse complement
ttgtggagggatttcattgg

SEQ ID NO: 5 - RNA, 20 nt, synthetic construct
  source 1..20: mol_type = other RNA
  misc_feature 1..20: note = IL2Rg gRNA1
gaaccctaccagtttctcat

SEQ ID NO: 6 - RNA, 20 nt, synthetic construct
  source 1..20: mol_type = other RNA
  misc_feature 1..20: note = IL2Rg gRNA1 reverse complement
atgagaaactggtagggttc

SEQ ID NO: 7 - RNA, 20 nt, synthetic construct
  source 1..20: mol_type = other RNA
  misc_feature 1..20: note = IL2Rg gRNA2
agatcctgacttgtctaggc

SEQ ID NO: 8 - RNA, 20 nt, synthetic construct
  source 1..20: mol_type = other RNA
  misc_feature 1..20: note = IL2Rg gRNA2 reverse complement
gcctagacaagtcaggatct